(19) United States
(12) Patent Application Publication
(10) Pub. No.: US 2025/0223560 A1
(43) Pub. Date: Jul. 10, 2025

(54) NOX4 INHIBITOR COMPOSITIONS AND METHODS FOR REGENERATION OF DYSTROPHIC MUSCLE

(71) Applicants: FULCRUM THERAPEUTICS, INC., Cambridge, MA (US); UNIVERSITY OF FLORIDA RESEARCH FOUNDATION, INCORPORATED, Gainesville, FL (US)

(72) Inventors: Anthony ACCORSI, Cambridge, MA (US); Genevieve WILSON, Cambridge, MA (US); David W. HAMMERS, Gainesville, FL (US); Lee SWEENEY, Gainesville, FL (US)

(73) Assignees: FULCRUM THERAPEUTICS, INC., Cambridge, MA (US); UNIVERSITY OF FLORIDA RESEARCH FOUNDATION, INCORPORATED, Gainesville, FL (US)

(21) Appl. No.: 18/878,213

(22) PCT Filed: Jun. 28, 2023
(86) PCT No.: PCT/US2023/069220

§ 371 Date: Dec. 23, 2024

Related U.S. Application Data
(60) Provisional application No. 63/356,959, filed on Jun. 29, 2022.

(51) Int. Cl. C12N 5/077; A61K 31/7105; C07K 16/18; G01N 33/50

(52) U.S. Cl. CPC C12N 5/0653; A61K 31/7105; C07K 16/18; G01N 33/5005

(57) ABSTRACT

The instant disclosure relates to compositions and methods that include NOX4 inhibitors as therapeutic agents capable not only of reducing fibrosis in muscle tissues of subjects having or at risk of developing a muscular dystrophy but also of inducing muscle tissue regeneration, by a mechanism apparently involving activation of fibro-adipogenic progenitor (FAP) cells.

### CROSS REFERENCE TO RELATED APPLICATION

This application claims priority to U.S. Provisional Application No. 63/356,959, filed Jun. 29, 2022, entitled “NOX4 Inhibitor Compositions and Methods for Regeneration of Dystrophic Muscle.” The entire content of the aforementioned application is incorporated herein by reference.

### FIELD OF THE INVENTION

The current disclosure relates to NOX4 inhibitor compositions and methods. In particular, the disclosure relates to therapeutic use of NOX4 inhibitors to treat subjects having a disease or disorder characterized by muscular dystrophy, by promoting muscle regeneration via activation of fibro-adipogenic progenitor (FAP) cells, satellite cells and/or mesenchymal stem cells in a treated subject.

### SEQUENCE LISTING

The instant application contains a Sequence Listing which has been filed electronically in eXtensible Markup Language (XML) format and is hereby incorporated by reference in its entirety. Said XML copy, created on Jun. 26, 2023, is named BN00028_0031_SL.xml and is 9,913 bytes in size.

### BACKGROUND OF THE INVENTION

Muscular dystrophies (MDs) are genetic muscle diseases characterized by progressive skeletal muscle degeneration and replacement of functional musculature with an aberrant fibrotic extracellular matrix (ECM). During the course of disease progression, MD patients exhibit a profound expansion of a fibrotic and fatty ECM as muscle fibers are lost. Muscular dystrophy refers to a group of more than 30 inherited diseases that cause muscular weakness. They are classified into nine categories and include Duchenne and Becker, Gaucher disease, Hurler's disease, Duchenne Muscular Dystrophy, Becker Muscular Dystrophy, Congenital Muscular Dystrophy, Myotonic Muscular Dystrophy, Limb-Girdle Muscular, Dystrophy Facioscapulohumeral Muscular Dystrophy, Emery-Dreifuss Muscular Dystrophy, and Distal Muscular Dystrophy.

There is therefore a previously unmet need in the art for therapeutics capable of slowing or reversing MDs, particularly therapeutics useful for disease management of older MD patients.

### BRIEF SUMMARY OF THE INVENTION

The instant disclosure is based, at least in part, upon identification of the ability of a NOX4 inhibitor (NOX4i) not only to reduce connective tissue fibrosis in skeletal muscle tissues of subjects having muscular dystrophy (MD), but also remarkably to promote activation of fibro-adipogenic progenitor (FAP) cells to induce muscle regeneration in such NOX4i-treated subjects. The instant disclosure therefore provides compositions and methods involving NOX4 inhibitor compounds capable of treatment of a subject having or at risk of developing a disease or disorder characterized by dystrophic muscles, including, e.g., a subject having MD. Muscle strength and/or conditioning can be monitored in a NOX4i-treated subject to assess treatment efficacy, or biopsy-based analyses of muscle regenerative markers (e.g., MYH3, UTRN, Pax7, MyoD) can be used to assess muscle regeneration in a treated subject in a clinical setting.

In one aspect, the instant disclosure provides a method for activating fibro-adipogenic progenitor (FAP) cells to increase muscle regeneration in a subject, the method involving administering a NADPH oxidase-4 (NOX4) inhibitor to the subject in an amount sufficient to activate FAP cells of the subject to increase muscle regeneration in the subject.

In one embodiment, the method further involves monitoring FAP cell-mediated muscle regeneration in the subject. Optionally, monitoring of FAP cell-mediated muscle regeneration in the subject includes measurement of one or more biomarkers for muscle regeneration in the subject; assessment of lean muscle volume in the subject; and/or assessment of fibrofatty infiltrate (e.g., muscle fat infiltration, fibrosis, etc.) in the subject. Optionally, the assessment of lean muscle volume in the subject is via magnetic resonance imaging (MRI); the assessment of fibrofatty infiltrate in the subject is via MRI or multispectral optoacoustic tomography; and/or the one or more biomarkers for muscle regeneration are selected from among myogenic transcription factors (e.g., MyoD, Myf5, MyoG), satellite cell factors (e.g., Pax7), and developmental myosin heavy chains (e.g., MYH3/MYH8).

In certain embodiments, the NOX4 inhibitor is a small molecule, a peptide/nucleic acid aptamer, an antibody (or antibody fragment) of a NOX4 interaction partner which behaves as an antagonist of NOX4 function, and/or an inhibitory and/or antisense RNA (e.g. siRNA, shRNA, ASO, etc.).

In a related embodiment, the small molecule inhibitor of NOX4 is steanaxib (GKT137831), GKT136901, GLX351322, APX-115, compound 7c, VAS2870, GLX481372, GLX7013114, UANox048, Ex. 101, thioridazine, prochlorproazine, chlorpromazine, fluphenazine, perhenazine, promazine, VAS3947, perhexiline, suramin, ebselin, celastrol, ML090, imipramin blue, imipramin hydrochloride, 3-methyl-1-phenyl-pyrazoline-5-one, GSK2795039, diphenyleneiodonium chloride (DPI), and/or a sulfonylurea compound. Optionally, the small molecule inhibitor of NOX4 is administered in combination with a nucleic acid therapeutic. Optionally, the nucleic acid therapeutic is a NOX4-targeting nucleic acid therapeutic.

A further aspect of the instant disclosure provides a pharmaceutical composition for inducing muscle regeneration in a subject, the pharmaceutical composition having a NOX4 inhibitor in an amount sufficient to induce muscle regeneration in the subject. In a related embodiment, the NOX4 inhibitor is present at a concentration capable of preventing at least 50% of myofibroblast differentiation in a subject administered the pharmaceutical composition, as compared to an appropriate control. Optionally, the appropriate control includes in vitro assessment of myofibroblast differentiation.

In another embodiment, the NOX4 inhibitor is present at a concentration capable of inducing at least 50% greater myogenic differentiation in a subject administered the pharmaceutical composition, as compared to an appropriate control. Optionally, the appropriate control includes in vitro assessment of myogenic differentiation.

Another aspect of the instant disclosure provides a method for treating a muscle in a subject, the method involving administering a NOX4 inhibitor to the subject in an amount sufficient to promote muscle regeneration in the subject, thereby treating the muscle in the subject.

An additional aspect of the instant disclosure provides a method for promoting muscle regeneration in a muscle of a subject, the method involving administering a NOX4 inhibitor to the subject in an amount sufficient to promote muscle regeneration in the subject.

A further aspect of the instant disclosure provides for use of a NOX4 inhibitor in the preparation of a medicament for inducing muscle regeneration in a subject, where the medicament is prepared to be administered in an amount sufficient to induce muscle regeneration in the subject by activating FAP cells of the subject.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, “administration” to a subject may include topical administration, parenteral administration, optionally for intravenous injection, inhalation, intravenous, intra-arterial, intratracheal, or involve direct injection into a tissue.

The term “treating” includes the administration of compositions to prevent or delay the onset of the symptoms, complications, or biochemical indicia of a disease (e.g., cancer, including, e.g., tumor formation, growth and/or metastasis), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

The term “pharmaceutically acceptable carrier” refers to a carrier for administration of a therapeutic agent. Such carriers include, but are not limited to, saline, buffered saline, dextrose, water, glycerol, ethanol, and combinations thereof.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawing, in which:

FIGS. 1A-1E show that NOX4 was increased in dystrophic skeletal muscle. FIG. 1A is a heat map of 26 fibrosis-associated genes that were identified as significantly upregulated in D2.mdx mouse quadriceps muscle (D2.mdx mice have been characterized as a preclinical model of the skeletal muscle pathology associated with Duchenne muscular dystrophy), relative to wild-type DBA/2J (D2.WT) mouse values (p<0.05). NOX4 transcript is highlighted with a box. The majority of such fibrosis-associated genes were extracellular matrix (ECM) components, including ten isoforms of collagen, four members of matrix metallopeptidases (MMPs; a class of matrix remodeling enzymes), the collagen crosslinking enzymes Lox and Loxl1, and other ECM-related genes, including Fn1 (fibronectin), Ltbp2, Postn (periostin), and Spp1 (osteopontin). Identified fibroblast-lineage markers included Pdgfra (platelet-derived growth factor receptor (PDGFR) a), Ctgf, Vim (vimentin), S100a4, and Acta2 (α-smooth muscle actin, aka SMA). FIG. 1B shows immunofluorescent detection of NOX4 in the gastrocnemius muscle. NOX4 was upregulated in D2.mdx mice, as compared to D2.WT mice. Images show immunofluorescence of NOX4/laminin, NOX4 alone and laminin alone. Scale bars represent 100 μm. FIG. 1C shows the immunofluorescent detection and upregulation of NOX4 in the diaphragm muscles of D2.mdx mice, compared to D2.WT mice. Images show immunofluorescence of NOX4/laminin, NOX4 alone and laminin alone, respectively. Scale bars represent 100 μm. FIG. 1D shows immunofluorescent detection of NOX4 in the vastus lateralis of DMD patients, which revealed NOX4 to be primarily localized to interstitial cells of dystrophic muscle. Images show immunofluorescence of NOX4/laminin, NOX4 alone and laminin alone, respectively. Scale bars represent 50 μm. FIG. JE shows that NOX4 was localized to fibroblast-lineage cells, which were also labeled by PDGFRα, in the dystrophic muscle of D2.mdx mice. Scale bars represent 50 m. Images show immunofluorescence of NOX4/PDGFRα, NOX4 alone, and PDGFRα alone.

FIGS. 2A-2E show that targeting NOX 4 reduced fibrosis in dystrophic muscle. FIG. 2A shows representative picrosirius red staining of diaphragm and gastrocnemius muscles in 6 month-old D2.WT (n=6), NOX4 wild-type (NOX4-WT) and knockout (NOX4-KO) littermates from a NOX4 KO line generated on the D2.mdx background (Nox4KO:mdx; n=12), as well as in D2.mdx mice that received vehicle or GK831 (GKT or GKT831, a NOX1/4 inhibitor) treatments beginning at 3 months of age (n=12-14). Fibrosis quantifications for 3-month-old D2.mdx mice are included to show baseline values for GKT treatment groups. FIG. 2B shows diaphragm fibrosis (% muscle area) measured via image quantification in 6 month-old D2.WT (n=6), D2.mdx NOX4 wild-type (NOX4-WT) and D2.mdx NOX4 knockout (NOX4-KO) littermates from a NOX4 KO line generated on the D2.mdx background (Nox4KO:mdx; n=12), as well as results for D2.mdx mice that received vehicle or GK831 (GKT) treatments beginning at 3 months of age (n=12-14). Percent values indicate the difference between mean values of the two groups. Data were analyzed using ANOVA followed by Tukey post-hoc tests (α=0.05; effect size is reported as η2) FIG. 2C shows fibrosis in gastrocnemius muscles (% muscle area) measured via image quantification in 6 month-old D2.WT (n=6), D2.mdx NOX4 wild-type (NOX4-WT) and D2.mdx NOX4 knockout (NOX4-KO) littermates from a NOX4 KO line generated on the D2.mdx background (Nox4KO:mdx; n=12), as well as results for D2.mdx mice that received vehicle or GK831 (GKT) treatments beginning at 3 months of age (n=12-14). Percent values indicate the difference between mean values of the two groups. Data were analyzed using ANOVA followed by Tukey post-hoc tests (α=0.05; effect size is reported as v12). FIG. 2D shows a graph of diaphragm muscle function, as assessed by measuring specific tension (N/cm³), in NOX4-WT (+/+) and NOX4-KO (−/−) littermates of the Nox4^{KO}:mdx mouse line. Data are presented as box-and-whisker plots with error bars representing minimum and maximum values. Data were analyzed using unpaired, two-tailed Welch's T-tests (α=0.05; effect size is reported as Cohen's d); *p<0.05 vs. 592 D2.WT values; #p<0.05 vs. 3 mo D2.mdx values; $p<0.05 vs. respective control group values. FIG. 2E shows a graph of extensor digitorum longus (EDL) muscle function, as assessed by measuring EDL maximum force (mN) in NOX4-WT (+/+) and NOX4-KO (−/−) littermates of the Nox4^{KO}:mdx mouse line. Data are presented as box-and-whisker plots with error bars representing minimum and maximum values. Data were analyzed using unpaired, two-tailed Welch's T-tests (α=0.05; effect size is reported as Cohen's d); *p<0.05 vs. 592 D2.WT values; #p<0.05 vs. 3 mo D2.mdx values; $p<0.05 vs. respective control group values. FIG. 2F shows biochemical quantification of collagen content, performed on gastrocnemius muscles of vehicle- and GKT-treated D2.mdx mice. Data are presented as box-and-whisker plots with error bars representing minimum and maximum values. Data were analyzed using unpaired, two-tailed Welch's T-tests (α=0.05; effect size is reported as Cohen's d); *p<0.05 vs. 592 D2.WT values; #p<0.05 vs. 3 mo D2.mdx values; $p<0.05 vs. respective control group values.

FIGS. 3A-3E show that targeting of NOX4 with the GKT831 NOX1/4 inhibitor reduced myofibroblasts and increased regeneration in dystrophic muscle. Data are displayed as mean±SEM and were analyzed using unpaired, two-tailed Welch's T-tests (α=0.05; effect size is reported as Cohen's d) or ANOVA followed by Tukey post-hoc tests (α=0.05; effect size is reported as η2). *p<0.05 vs. D2.WT values; #p<0.05 vs. respective control group values. Unless otherwise indicated, scale bars indicate 50 m. FIG. 3A shows representative immunofluorescent images and quantification of myofibroblast prevalence in gastrocnemius muscles of D2.WT, NOX4-WT (+/+) and NOX4-KO (−/−) mice of the Nox4^{KO}:mdx mouse line, as determined by co-labeling with PDGFRα, vimentin, smooth muscle actin (SMA) and DAPI staining (arrows mark myofibroblasts; BV=blood vessel). Pax7 immunofluorescence was performed to assess satellite cell numbers. FIG. 3B shows representative immunofluorescent images and quantification of muscle periostin content in D2.WT, NOX4-WT (+/+) and NOX4-KO (−/−) mice the Nox4^{KO}:mdx mouse line. Pax7 immunofluorescence was performed to assess satellite cell numbers. Populations of myofiber-associated and interstitial Pax7⁺ cells were identified and investigated for impact of NOX4-targeting, as the GKT NOX1/4 inhibitor significantly reduced periostin⁺ area in GKT-treated mice. FIG. 3C shows representative images and quantification of cell populations (arrows mark Pax7⁺ cells) of both myofiber-associated and interstitial Pax7⁺ cells that were identified as affected by GKT-mediated NOX4 targeting/inhibition. FIG. 3D shows evidence of restarted muscle regeneration in the gastrocnemius muscle induced by NOX4 targeting, as shown by the increased number of fibers in GKT-treated muscle that exhibited centrally-located nuclei FIG. 3E shows staining for myosin X (Myo10; stars indicate fibers marked as recently regenerated), which also yielded evidence of restarted muscle regeneration via inhibition of NOX4.

FIGS. 4A and 4B show that short-term NOX4 inhibition induced myofibroblast apoptosis. Six month-old male D2.mdx mice were treated with vehicle or 60 mg/kg GKT831 (GKT) for seven days (n=6). Data are displayed as mean±SEM and were analyzed using unpaired, two-tailed Welch's T-tests (α=0.05; effect size is reported as Cohen's d). FIG. 4A shows apoptosis of PDGFRα⁺ cells in vehicle- and GKT-treated gastrocnemius muscles, as assessed by immunofluorescent detection of active caspase-3. FIG. 4B shows measured gene expression for Col1a2, Acta2, Il6, and Tgfb1 in gastrocnemius muscles, assessed using real-time PCR.

FIGS. 5A-5D depict NOX4 expression in dystrophic muscle, supplementary to FIGS. 1B-1E above. FIG. 5A shows representative immunoblots that displayed differential content of dystrophin, fibronectin, and NOX4 protein levels in quadriceps lysates from 4 month male D2.WT and D2.mdx mice (n=4). NOX4 immunoblotting data were quantified, normalized to GAPDH signal, and displayed relative to D2.WT values. Data are presented as box-and-whisker plots, with error bars representing minimum and maximum values, and were analyzed using unpaired, two-tailed Welch's T-tests (α=0.05; effect size is presented as Cohen's d). Scale bar represents 100 km. FIG. 5B shows immunofluorescent detection of myofibroblasts in D2.mdx gastrocnemius muscles using antibodies against PDGFRα, vimentin, SMA, and DAPI. FIG. 5C shows that NOX4 localized to PDGFRα⁺ cells in the LGMD2B mouse model of muscular dystrophy (9 month-old A/J mice). FIG. 5D shows that NOX4 also localized to PDGFRα⁺ cells in the LGMD2F mouse model of muscular dystrophy (3 month-old Sgcd^{-/-} mice). Scale bars represent 50 μm.

FIGS. 6A-6D show that NOX4 ablation did not affect the early time course of muscle fibrosis, supplementary to FIGS. 2A-2C above. FIG. 6A shows representative immunofluorescent images of Nox4^{WT}:mdx and Nox4^{KO}:mdx gastrocnemius (gastroc) muscles stained with anti-NOX4 antibody. FIG. 6B show representative images of histological assessment of muscle fibrosis in the diaphragm and gastrocnemius muscles of 3 month-old Nox4^{WT}:mdx and Nox4^{KO}:mdx mice using picrosirius red staining (n=4). No significant differences in muscle fibrosis between the groups at this age were identified. Scale bar represents 100 μm. FIG. 6C shows histological quantifications of diaphragm fibrosis that also revealed no significant differences between the groups at this age. Data are presented as box-and-whisker plots with error bars representing minimum and maximum values. Data were analyzed using unpaired, two-tailed Welch's T-tests (α=0.05; effect size is presented as Cohen's d). Scale bar represents 100 m. FIG. 6D shows additional histological quantifications of gastrocnemius fibrosis that also revealed no significant differences between the groups at this age. Data are presented as box-and-whisker plots with error bars representing minimum and maximum values. Data were analyzed using unpaired, two-tailed Welch's T-tests (α=0.05; effect size is presented as Cohen's d). Scale bar represents 100 μm.

FIG. 7 shows IC50 curve results obtained for exemplary NOX4 inhibitory compounds FTX7995, FTX1620, FTX 7981, FTX7983, and FTX7974, which were assessed for potency of decreasing myofibroblast content in treated cells and for the IC50 of such effect.

FIG. 8 shows a second round of IC50 curve results obtained for exemplary NOX4 inhibitory compounds FTX7995, FTX1620, FTX 7981, FTX7983, FTX7974, and FTX8018, which were assessed for potency of decreasing myofibroblast content in treated cells and for the IC50 of such effect.

FIG. 9 shows panels of fluorescent images for respective fibrogenic induction media (FIM, basal media plus 5-10 ng/mL TGFβ) control treatment, FTX001620, FTX008081, FTX007963, FTX007974 and FTX007995 treatments (all NOX4 inhibitors at 1 μM), respectively stained with DAPI, assessed for α-SMA (immunofluorescence), and stained with phalloidin, which revealed that each of the NOX4 inhibitors restored NOX4i-treated mesenchymal stem cells to a cellular phenotype predicted to promote myogenesis (i.e., muscle regeneration via FAP cells) when administered to a subject in vivo, as indicated by prevalent actin fluorescence, including F-actin (phalloidin staining).

FIG. 10 shows that mesenchymal stem cells treated with 10 nM, 100 nM and 1 μM of NOX4-inhibitory compound FX007995 exhibited α-SMA immunofluorescence and phalloidin (F-actin) staining at all treated concentrations, which demonstrated a reduction of myofibroblast differentiation in such treated mesenchymal stem cells. Reduction of myofibroblast differentiation is predictive of myogenesis promotion when cultured with (or in communication with) muscle progenitors (as seen for D2-mdx data presented elsewhere herein). These data are again indicative of restoration of NOX4i-treated mesenchymal stem cells to a cellular phenotype predicted to promote myogenesis (restoration of a pro-regenerative phenotype, i.e., muscle regeneration via FAP cells) when administered to a subject in vivo. FIM images are shown as a positive control for filamentous actin formation.

FIG. 11 shows the NOX4 inhibitor compound FTX001620 reduced the myofibroblast content of treated cells in a dose-responsive manner, in contrast to each of respective negative/redox active control compounds FTX007991, FTX006277, FTX002775, FTX007678, FTX007876, FTX007578, and FTX002278.

FIG. 12 shows that negative/redox active compounds FTX007991, FTX007678, FTX002775, and FTX006277 did not show significant phalloidin (F-actin) staining at 1 M, as compared to untreated cells as a negative control and FIM cells. All cells were stained with DAPI, assessed for α-SMA via immunofluorescence, and stained with phalloidin, respectively.

FIG. 13 shows the IC50 curve results obtained for exemplary NOX4 inhibitory compounds FTX001620, FTX7990, FTX007971, FTX007972, and FTX007973, which were assessed for potency of decreasing myofibroblast content in treated cells and for the IC50 of such effect.

FIG. 14 shows replicate IC50 curve results obtained for exemplary NOX4 inhibitory compounds FTX001620, FTX7990, FTX007971, FTX007972, and FTX007973, which were assessed for potency of decreasing myofibroblast content in treated cells and for the IC50 of such effect.

FIG. 15 shows that NOX4 inhibitor compounds FTX001620, FTX007981, FTX007983, FTX007974, and FTX007995 exhibited both α-SMA immunofluorescence and phalloidin (F-actin) staining at 1 μM, which provided further evidence of the reduction in myofibroblast differentiation observed in such NOX4i-treated cells and was again indicative of restoration of NOX4i-treated mesenchymal stem cells to a cellular phenotype predicted to promote myogenesis (restoration of a pro-regenerative phenotype, i.e., muscle regeneration via FAP cells) when administered to a subject in vivo.

FIGS. 16A and 16B show that the DPI NOX4 inhibitor promoted myogenic differentiation of myoblasts in myoblast-FAP co-cultures, as compared to control-treated co-cultures. FIG. 16A shows the effect of FAP-myoblast co-culture at three days of differentiation in the presence of either vehicle (DMSO) alone (control, top) or 0.1 μM DPI (bottom), where FAP cells constitutively expressed td‘l’omato (red fluorescence). Significantly greater levels of the myogenic differentiation marker, myosin heavy chain (MHC), were observed in DPI-treated myoblasts in co-culture, as compared to vehicle-treated myoblasts in co-culture. DAPI was used to stain nuclei of all cells in co-culture. Scale bars represent 100 μm. FIG. 16B shows a plot of observed levels of MHC-positive myocytes in either vehicle-treated co-cultures or DPI-treated co-cultures, with MHC-positive myocytes quantified as number of cells per mm².

### DETAILED DESCRIPTION OF THE INVENTION

The instant disclosure provides, at least in part, compositions and methods that include NOX4 inhibitors as therapeutic agents not only capable of reducing and/or preventing ongoing fibrosis in a subject having or at risk of developing a disease or disorder characterized by dystrophic muscle, but also capable of positively stimulating muscle regeneration, restoring cells to a pro-regenerative phenotype, apparently via a fibro-adipogenic progenitor (FAP) cell-mediated mechanism.

Muscular dystrophies (MDs) are progressive muscle diseases. Therapeutics capable of combating the severe fibrosis that replaces functional muscle in these devastating diseases is a major unmet clinical need, particularly for the treatment of older patients. The current disclosure reveals that targeting NOX4 in dystrophic muscle promotes the beneficial remodeling of disease-burdened musculature. This is achieved by the removal of disease-causing cells, known as myofibroblasts, which results in reduced muscle fibrosis and rejuvenation of muscle regeneration. Remarkably, such rejuvenation of muscle regeneration has been identified herein to be promoted by NOX4 inhibitory compounds which restore cells within targeted muscle to a pro-regenerative phenotype, apparently via activation of fibro-adipogenic progenitor (FAP) cells, which induce regrowth of skeletal muscle in particular. NOX4-targeting strategies, therefore, represent therapeutics capable of improving muscle disease caused by muscular dystrophy, as well as other muscle pathologies.

Muscular dystrophies are also genetic muscle diseases that result in progressive muscle degeneration followed by the fibrotic replacement of affected muscles as regenerative processes fail. Therapeutics that specifically address the fibrosis and failed regeneration associated with MDs represent a major unmet clinical need for MD patients, particularly those with advanced stage disease progression. The current disclosure identifies targeting NAD(P)H oxidase (NOX) 4 (NOX4) as a strategy to reduce fibrosis and promote regeneration in disease-burdened muscle, e.g., that models Duchenne muscular dystrophy (DMD). NOX4 is elevated in the muscles of dystrophic mice and DMD patients, localizing primarily to interstitial cells located between muscle fibers. Genetic and pharmacological targeting of NOX4 was identified to significantly reduce fibrosis in dystrophic respiratory and limb muscles. Without wishing to be bound by theory, mechanistically, NOX4 targeting decreased the number of fibrosis-depositing cells (myofibroblasts) and, remarkably, restored the number of muscle-specific stem cells (satellite cells) to their physiological niche, thereby rejuvenating muscle regeneration. Furthermore, acute inhibition of NOX4 was sufficient to induce apoptotic clearing of myofibroblasts within dystrophic muscle. These data have identified that targeting NOX4 for inhibition/knockdown is an effective strategy to promote the beneficial remodeling of disease-burdened muscle representative of DMD and, likely, other MDs and muscle pathologies.

The progressive replacement of contractile muscle tissue with non-contractile connective tissue, fat and fibrosis, are causal drivers of loss of function in patients with muscular dystrophies. Inherent to this progressive replacement is a failure of normal muscle regeneration. Re-balancing repair and replacement, therefore, serves as a rational therapeutic basis for extending the healthy lifespan of muscle in dystrophic disorders.

Fibro-adipogenic progenitor cells (FAP cells) have previously been shown to play critical roles in muscle homeostasis and disease. FAP cells facilitate muscle regeneration in acute injury settings but also differentiate into the pathogenic replacement cell types (myofibroblasts and adipocytes). Based on these known functions, they serve as a high value cell type relevant to rebalancing repair and replacement in dystrophic muscle.

NOX4 has been previously identified to inhibit fibrosis in other organs; however, NOX4 has not been tested in the context of skeletal muscle with NOX4 inhibitors until the instant disclosure. NOX4 inhibition was tested herein in the context of dystrophic muscle as a means to reduce muscle replacement. It was identified herein that not only does NOX4 inhibition remodel existing fibrosis and prevent ongoing fibrosis, but also stimulates muscle regeneration via promotion of satellite cell-mediated repair, a previously unknown outcome of NOX4 inhibition. Therefore, reducing connective tissue in muscle is not in and of itself beneficial to a subject—muscle also needs to repair itself to form a functional syncytium. Treatment of a subject with a NOX4 inhibitor achieves both.

The identification of treatments that effectively improve dystrophic muscle has been previously hindered by limited knowledge of the cellular mechanisms responsible for the development of muscle fibrosis and impairment of muscle regeneration associated with the disease. Under normal conditions, skeletal muscle displays robust regeneration following injury, which is largely mediated by muscle-resident stem cells, known as satellite cells (5, 6). The process of muscle regeneration also requires an array of chemical and physical cues provided by immune cells, myogenic cells, fibroblasts, and other cellular populations (7-9). These factors are temporally orchestrated to form new muscle that is accommodated with adequate vasculature, innervation, and ECM structural support, leading to resolution of the injury response and return to homeostasis (5, 7, 10). Indeed, perturbations to any of these components contributes to maladaptive muscle regeneration (7, 9, 11, 12). In dystrophic muscle, the continuous and asynchronous combination of degeneration and regenerative processes within the same muscle disrupts the timing of these events (13).

This ultimately leads to regenerative impairments and progressing fibrosis that characterize the disease burden of late-stage dystrophic muscle. Upregulation of NAD(P)H oxidase (NOX) 4 was previously identified in diseased muscle of D2.mdx mice, a severe mouse model of DMD (14-16), using a previously published transcriptomic data set (17). NOX4 is a reactive oxygen species (ROS)-generating enzyme that has been implicated in fibrosis of the lung, kidney, liver, and heart (reviewed in (18)). While elevated Nox4 expression has been noted in dystrophic mouse hearts (19), NOX4 has not been previously studied in regard to skeletal muscle fibrosis. The current disclosure initially determined if the targeting of NOX4 was an effective strategy to prevent fibrosis and enhance regeneration in dystrophic muscle. Herein, it has been shown that NOX4 localizes primarily to interstitial cells of dystrophic muscle that resemble active ECM-secreting cells, known as myofibroblasts, which are typically only found in damaged or diseased tissue (20). The targeting of NOX4 both by genetic ablation and pharmacological inhibition promoted the beneficial remodeling of diseased muscle by reducing muscle fibrosis. Importantly, NOX4 targeting substantially reduced myofibroblasts within disease-burdened muscle, restored the localization of satellite cells to their physiological niche, and increased evidence of muscle regeneration. These data implicated NOX4 in the development of MD-associated skeletal muscle pathology, and demonstrated that targeting NOX4 for inhibition was an effective strategy to promote beneficial remodeling of dystrophic muscle.

Identification of therapeutics capable of remodeling the disease-burdened muscle of MD patients has to date been a major unmet clinical need. Discovery and advancement of such therapeutics is particularly important for the treatment of older DMD patients who have undergone substantial replacement of muscle with pathological ECM. The current disclosure has identified NOX4 as an efficacious target to promote the remodeling of dystrophic muscle by preventing fibrosis and enhancing regeneration. This was achieved using genetic ablation and, importantly, pharmacological inhibition with a clinical-stage drug. The efficacious effect of NOX4 targeting is primarily associated with a reduction of myofibroblasts in dystrophic muscle. This clearance of myofibroblasts appears to alleviate both the pro-fibrotic and anti-regenerative environment associated with disease-burdened muscle. These findings indicate that NOX4-targeting interventions represent remodeling therapeutics capable of improving the muscle disease state in DMD and other muscle diseases.

Myofibroblasts are specialized ECM-depositing cells that are activated to facilitate tissue repair; however, they contribute to tissue pathology in chronic diseases by producing excessive ECM, thereby, causing progressive fibrosis (20). A major finding of the instant disclosure is that the beneficial remodeling incurred by NOX4 targeting involved the clearance of myofibroblasts within the muscles. While similar results have been shown in lung fibrosis (24, 43), there appears to be no prior report of a treatment that primarily targets myofibroblasts in dystrophic muscle. The clearing of myofibroblasts by targeting NOX4 is a two-hit approach to promote beneficial remodeling of dystrophic muscle, as a) the cellular source of fibrosis is removed from the system, and b) negative regulation of regenerative efforts is alleviated, thereby allowing a stalled myogenic program to proceed.

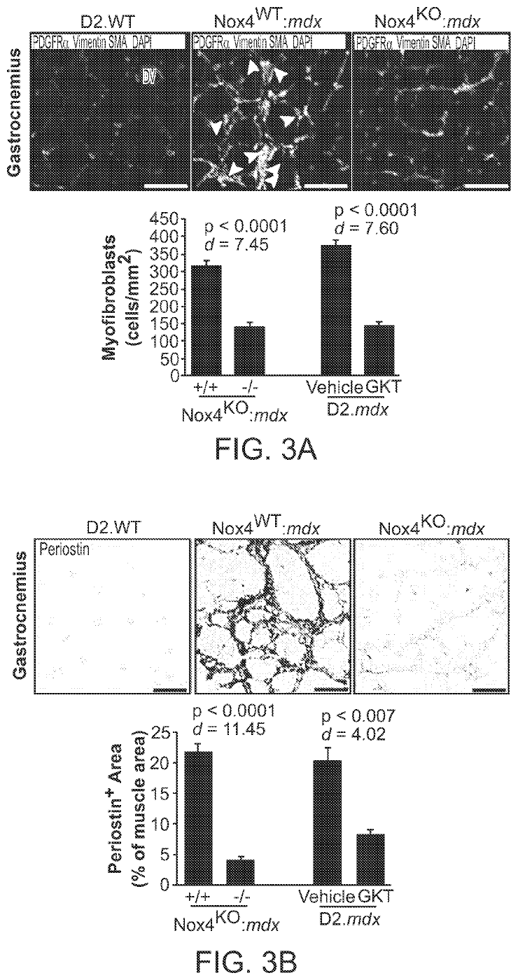

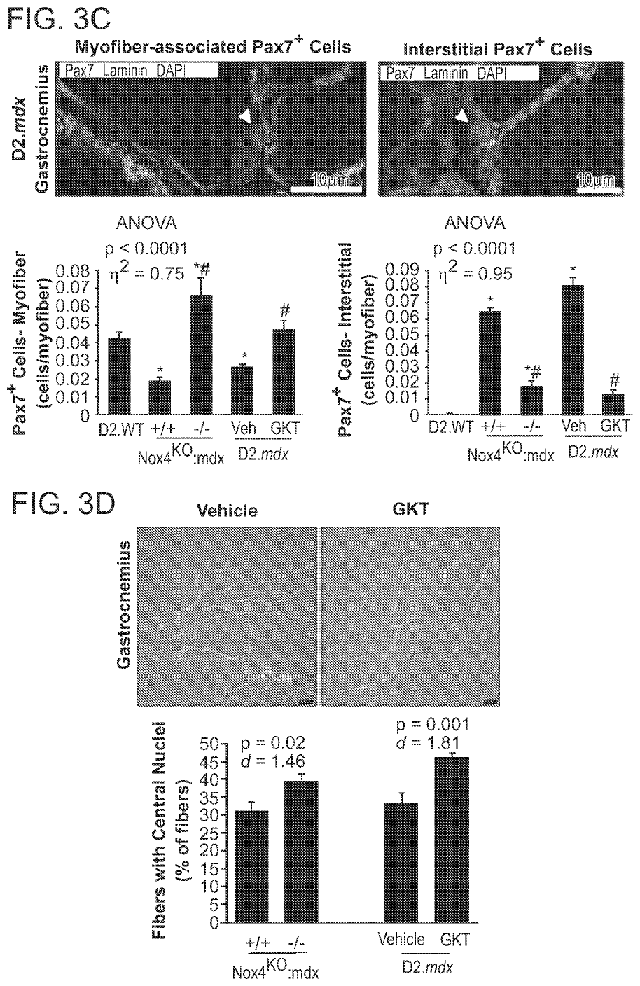

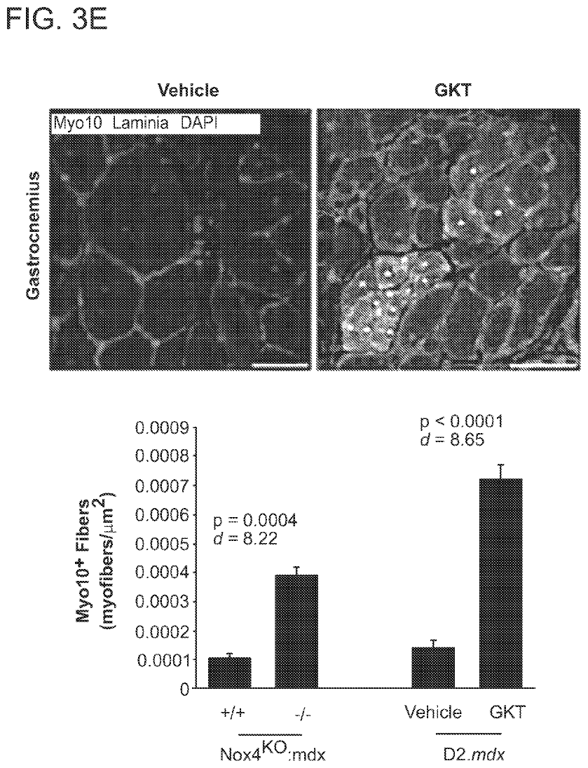

The inhibitory effect of myofibroblasts on muscle regeneration may be mediated by multiple mechanisms. For instance, periostin, a myofibroblast-specific protein that is secreted during ECM production (36), appears to directly inhibit muscle regeneration (37), and its ablation enhances regeneration (18, 38). Similarly, NOX4 inhibition drastically reduced periostin content in dystrophic muscle (FIG. 311), which coincided with evidence of rejuvenated regeneration (FIGS. 3D-3E). Furthermore, myofibroblasts likely also release signaling molecules that impede satellite cell function, such as TGFβ (44, 45). In the instant disclosure, many Pax7⁻ cells were found to be mislocalized to the interstitium of dystrophic muscle (FIG. 3C), which were largely restored to a myofiber-associated localization upon the removal of myofibroblasts by NOX4 targeting. Observed displacement of Pax7⁺ cells to the interstitium of dystrophic muscle was similar to results previously shown in DMD patient muscle (40) and murine cancer cachexia (41). Such results indicated that myofibroblasts, whether by secreted factors or physical interactions, essentially halted the activity of satellite cells within dystrophic muscle, thereby contributing to the regenerative decline associated with DMD. The arrest of satellite cell activity appeared to be reversible, as was previously shown in cancer cachexia (41), which allowed active regeneration of dystrophic muscle to be restarted upon alleviation of these inhibitory signals.

Currently, the landscape of DMD therapeutics is at a pivotal crossroad. Advancements in gene therapy have led to clinical trials evaluating the delivery of miniaturized dystrophin transgenes to the muscles of DMD patients (46). If successful, these therapies will transform DMD into a slower progressing disease that still exhibits progressive replacement of muscle with ECM, which is seen in Becker muscular dystrophy patients (47, 48). Furthermore, it is not clear how pre-existing disease burden will affect the delivery or efficacy of these gene therapies, particularly in the case of treating older DMD patients. Thus, therapeutics capable of remodeling disease-burdened muscle will remain a major clinical need for the management of DMD, whether as monotherapies or in combination with gene therapy. The current disclosure indicates that NOX4-inhibiting strategies represent effective remodeling therapeutics capable of reducing fibrosis and enhancing muscle regeneration in dystrophic muscle through the targeting of myofibroblasts. Such findings have direct application to the treatment of both genetic and non-genetic muscle diseases that exhibit progressive fibrosis and failed regeneration.

### NOX4 Inhibitors

Certain aspects of the instant disclosure feature NOX4 inhibitory compounds. Exemplary NOX4 inhibitors include the following, without limitation.

Setanaxib (aka GKT137831/FTX001620) is an experimental, orally bioavailable dual inhibitor of NADPH oxidase isoforms NOX4 and NOX1, additionally characterized as a NOX5 inhibitor, which possesses potential for redox cycling. Setanaxib is a member of the pyrazolopyridine dione chemical series. The chemical structure of Setanaxib is:

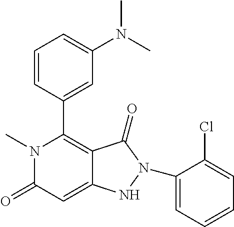 [SMILES: CN(C)c1cccc(-c2c3c(=O)n(-c4ccccc4Cl)[nH]c3cc(=O)n2C)c1]

GKT136901/FTX8018 is a NOX1 and NOX4 inhibitor that is orally bioavailable, which has been described in the art as possessing potential application in the areas of diabetic nephropathy, stroke, and neurodegeneration. GKT136901 possesses potential for redox cycling, and the compound acts as a selective scavenger of peroxynitrite (PON) in the submicromolar concentration range. The chemical structure of GKT136901 is:

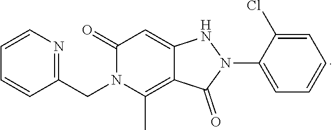 [SMILES: Cc1c2c(=O)n(-c3ccccc3Cl)[nH]c2cc(=O)n1Cc1ccccn1]

GLX351322/FTX007971 is a NOX 4 inhibitor that is orally bioavailable and described to possess moderate activity in cells at about 5 μM. GLX351322 is active in a stroke model for mice and has low potential for redox cycling. Additionally, GLX351322 treatment rescued synaptic and memory deficits, and decreased oxidative stress and amyloid levels in the hippocampus of APP/PS1 (amyloid precursor protein/presenilin-1) mice. The chemical structure of GLX351322 is:

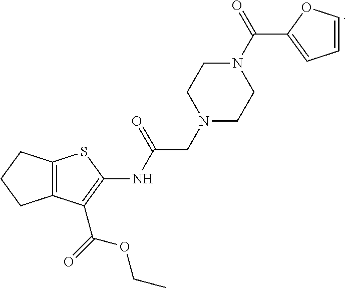 [SMILES: CCOC(=O)c1c(NC(=O)CN2CCN(C(=O)c3ccco3)CC2)sc2c1CCC2]

Compound 7c/FTX007990 is an orally bioavailable NOX4 inhibitor that has been described as active in vivo. A no adverse effect level (NOAEL) is defined as the highest dose where the effects observed in the treated group do not imply an adverse effect to the subject. In mice, the NOAEL for Compound 7c was 1000 mg/kg after 2 weeks. Compound 7c possesses potential for redox cycling. The chemical structure of Compound 7c is.

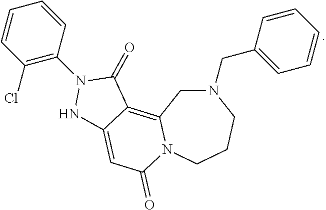 [SMILES: O=c1c2c3n(c(=O)cc2[nH]n1-c1ccccc1Cl)CCCN(Cc1ccccc1)C3]

VAS2870/FTX007972 is a cell-permeable and covalent NOX inhibitor that is not isoform-selective. In addition to NOX4 inhibition, VAS2870 is also known to suppress reactive oxygen species (ROS) production in several cell types. VAS2870 has been described to effectively impair cell growth and increase apoptosis induced by transforming growth factor β (TGF-β) in liver cancer cells. The chemical structure of VAS2870 is:

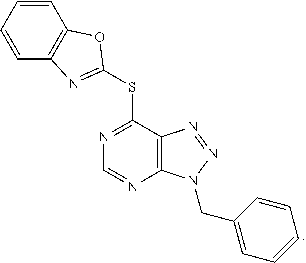 [SMILES: c1ccc(Cn2nnc3c(Sc4nc5ccccc5o4)ncnc32)cc1]

GLX481372/FTX007983 (WO2016133446) is a NOX4 and NOX5 inhibitor that has some selectivity against NOX1, NOX2 and NOX3. It has a low potential for redox cycling, The chemical structure of GLX481372/FTX007983 is:

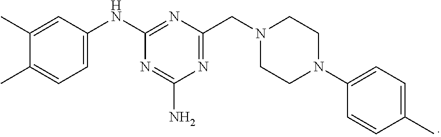

GLX7013114/FTX007981 (WO2019215291) is a NOX4 inhibitor with some reported selectivity toward NOX1, per measured IC50 values of 0.3 μM (IC50 NOX4) and 22 M (IC50 NOX1). GLX7013114 is active in cells and has a low potential for redux cycling. The chemical structure of GLX7013114 is:

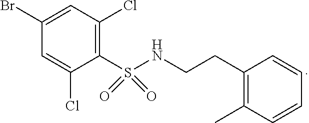 [SMILES: Cc1ccccc1CCNS(=O)(=O)c1c(Cl)cc(Br)cc1Cl]

UANox048/FTX007974 is a NOX4-selective compound that has been described as active in cells that are from 10 M to 30 μM in size. UANox048/FTX007974 is part of a group of NOX4 inhibitors developed for the treatment of fibrotic diseases. UANox048/FTX007974 has a low potential for redox cycling. The chemical structure of UANox048/FTX007974 is:

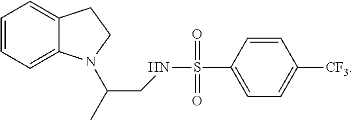 [SMILES: CC(CNS(=O)(=O)c1ccc(C(F)(F)F)cc1)N1CCc2ccccc21]

Ex. 101/FTX007995 (WO2017192304) is a compound that is strongly active in cells with a low potential for redox cycling. It is part of a group of NOX4 inhibitors developed for the treatment of fibrotic diseases such as sclerodermia, lung disease, heart disease, liver disease, kidney disease and other diseases with fibrosis, The chemical structure of FTX007995 is:

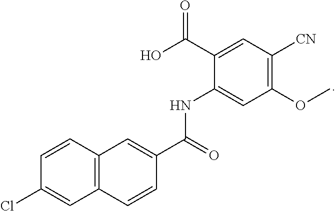 [SMILES: COc1cc(NC(=O)c2ccc3cc(Cl)ccc3c2)c(C(=O)O)cc1C#N]

In certain embodiments the instant disclosure provides that Nox4 inhibitors are capable of elevating levels of phalloidin staining (indicative of FAP selective activation/skeletal muscle regeneration) where fold increase is relative to untreated cells around the order of 50%, optionally up to 2-fold, up to 3-fold, up to 5-fold, up to 10-fold, up to 20-fold, up to 30-fold, and optionally up to 50-fold over untreated cells, relative to baseline staining of untreated or other appropriate control cells.

In other embodiments, NOX4 inhibitors of the instant disclosure activate smooth muscle at levels of approximately about 50% elevation, optionally up to 2-fold, up to 3-old, up to 5-fold, up to 10-fold, up to 20-fold, up to 30-fold, optionally up to 50-fold over untreated cells, relative to baseline staining of untreated cells and/or other appropriate control

### Methods of Treatment

In certain embodiments, the compositions of the instant disclosure can be used to treat or prevent a dystrophic muscle disease or disorder, including but not limited to Duchenne Muscular Dystrophy (DMD), Becker MD, congenital MD, distal MD, Emery-Dreifuss MD, Facioscapulohumeral MD, Limb-Girdle MD, myotonic MD, and Oculopharyngeal MD.

NOX4i compositions of the instant disclosure may be administered as first line treatments or as secondary treatments.

### Pharmaceutical Compositions

Agents of the present disclosure can be incorporated into a variety of formulations for therapeutic use (e.g., by administration) or in the manufacture of a medicament (e.g., for treating or preventing MD, e.g., DMD) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutical-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline. PBS. Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red, iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g., sodium or potassium salts; and alkaline earth metal salts, e.g., calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e g, at least National Food (NIL) grade generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as a NOX4 inhibitor, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e., having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymer and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III. CRC Press, Boca Raton, Fla., 1987, pp 137-149.

Pharmaceutical compositions of the present disclosure containing an agent described herein may be used (e.g. administered to an individual, such as a human individual, in need of treatment with a NOX4 inhibitor) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan, Animal experiments provide reliable guidance for the determination of effective doses for human therapy. Interspecies scaling of effective doses can be performed following the principles described in Mordenti, J. and Chappell, W “The Use of Interspecies Scaling in Toxicokinetics,” In Toxicokinetics and New Dug Development, Yacobi et al., Eds, Pergamon Press, New York 1989, pp. 42-46.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for oral delivery or intravenous injection to a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a NOX4 inhibitor) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

The agents or compositions can be administered in combination with additional pharmaceutical agents that improve their activity (e.g., activity (e.g., potency and/or efficacy) in treating a disease (e.g., MD) in a subject in need thereof, in preventing a disease in a subject in need thereof, in reducing the risk of developing a disease in a subject in need thereof, etc. in a subject or cell. In certain embodiments, a pharmaceutical composition described herein including an agent (e.g., a NOX4 inhibitor) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a microRNA or other nucleic acid therapeutic, an immunotherapy, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, other small molecules and/or nucleic acid therapeutics.

Dosages for a particular agent of the instant disclosure may be determined empirically in individuals who have been given one or more administrations of the agent.

Administration of an agent of the present disclosure can be continuous or intermittent, depending, for example, on the recipients physiological condition, whether the purpose of the administration is therapeutic or prophylactic, and other factors known to skilled practitioners. The administration of an agent may be essentially continuous over a Preselected period of time or may be in a series of spaced doses.

Guidance regarding particular dosages and methods of delivery is provided in the literature, see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Combination Therapies

In certain embodiments, an agent (e.g., a NOX4 inhibitor) or composition, as described herein, can be administered in combination with one or more additional pharmaceutical agents (e.g., therapeutically and/or prophylactically active agents), which are different from the agent or composition and may be useful as, e.g., combination therapies. Exemplary combination therapies contemplated for use with the NOX4 inhibitors of the instant disclosure include, without limitation, MiR-29 (an anti-fibrotic that inhibits collagen synthesis but does not alter TGF-beta1 levels and has been described as detrimental to the muscles of a treated subject) and halofuginone (a compound possessing anti-fibrotic activity via inhibiting collagen synthesis, thereby predicted to not be beneficial for restarting muscle regeneration in a treated subject.

Other combination therapies known to those of skill in the art can be used in conjunction with the compositions and methods of the instant disclosure.

### Kits

The instant disclosure also provides kits containing agents of this disclosure for use in the methods of the present disclosure. Kits of the instant disclosure may include one or more containers comprising an agent (e.g., a NOX4 inhibitor) of this disclosure and/or may contain agents (e.g., oligonucleotide primers, probes, etc.) for identifying MD and/or muscle regeneration in a subject. In some embodiments, the kits further include instructions for use in accordance with the methods of this disclosure. In some embodiments, these instructions comprise a description of administration of the agent to treat or diagnose, e.g., a MD, according to any of the methods of this disclosure. In some embodiments, the instructions comprise a description of how to detect MD in a subject and/or to assess FAP cell activation and/or muscle regeneration, for example in an individual, in a tissue sample, or in a population of cells. The kit may further comprise a description of selecting an individual suitable for treatment based on identifying whether that subject has a form of MD that is treatable with a NOX4 inhibitor.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a MD disease or disorder, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent in the composition is a NOX4 inhibitor. The container may further comprise a second pharmaceutically active agent (e.g., MiR-29 and/or halofuginone).

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.), Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Development of D2.mdx and D2.WT Mice and Evaluation

D2.mdx (Jax #013141) and D2.WT (Jax #000671) mice were originally obtained from Jackson Laboratory. The Nox4^{KO}:mdx mouse line was created by crossing the Nox4^{tm1Klr} knockout allele ((23); Jax #022996) onto the D2.mdx background for five generations, as previously reported (15). Following the second backcross, only Nox4^{tm1Klr/+} mice homozygous for the DBA/2J polymorphism of Libp4 were selected for continued line development (16). This is because both Nox4 and Ltbp4 are located on murine chromosome 7, and cross-over is required for both alleles to segregate together. At the completion of the backcrossing, Nox4 heterozygous breed pairs were mated to produce Nox4^{WT}:mdx and Nox4^{KO}:mdx littermates that were used in experiments. All mice were genotyped for Nox4 using primer sequences provided by Jackson Laboratory, and for Ltbp4 and mdx alleles using published genotyping primers (16).

Mice were housed 3-5 mice per cage, randomly assigned into groups, provided ad libitum access to food (NIH-31 Open formulation diet; Envigo #7917), water, and enrichment, and maintained on a 12-hour light/dark system. Once daily (Q.D.) GKT831 (purchased from Ambeed, Inc.) was administered orally (P.O.) suspended at a concentration of 36 mg/mL in a vehicle consisting of sterilized sunflower seed oil (Sigma-Aldrich). This method of drug delivery resulted in voluntary ingestion of administered solution by mice, similar to syrup-based vehicles as previously reported (17). The GKT831 treatment group received a dose of 60 mg/kg (21). All animal procedures were approved and conducted in accordance with the University of Florida IACUC and reported using the recommendations of ARRIVE guidelines.

### Immunofluorescence and Histological Evaluations

OCT-embedded murine tissues were cryo-sectioned at 10 m and fixed in 4% PFA. Immunofluorescent analysis was performed using anti-NOX4 (1:2000; Abcam #13303), anti-laminin (1:800; Novus #MAB2549), anti-vimentin (1:1000; Novus #MB300), anti-PDGFRα (1:500; R&D Systems #AF1062), anti-SMA (1:1000; Abcam #ab5694), anti-periostin (1:500, Abcam #ab14041), anti-Pax7 (1:100; R&D Systems #MAB1675), anti-Myosin X (1:800; Sigma #HPA024223) and anti-active caspase-3 (1:1000; Cell Signaling 49661) primary antibodies. Mouse tissue sections incubated with mouse monoclonal antibodies were first incubated with a solution containing donkey anti-mouse IgG AffiniPure Fab fragments (1:25 in PBS; Jackson ImmunoResearch #715-007-003) for one hour prior to blocking. Following overnight incubation in primary antibody, sections were rinsed with PBS and incubated with the appropriate species-specific secondary antibody (1:500 dilution). Lipofuscin-dependent autofluorescence was quenched using a 0.1% solution of Sudan black B following secondary antibody incubation. Slides were cover-slipped using Prolong Gold mounting reagent (Invitrogen). Images were acquired using a Leica SP8 confocal microscope. All comparative images were stained simultaneously and acquired using identical settings. Human samples were obtained through the National Disease Research Interchange (Philadelphia, PA).

Picrosirius red staining was performed as previously described (14) following decalcification of muscle sections using Formical-2000 (StatLab). Slides were visualized with a Leica DMR microscope, and images were acquired using a Leica DFC310FX camera interfaced with Leica LAS X software. All comparative images were stained simultaneously and acquired using identical settings. Images were processed and analyzed by investigators blinded to study groups using ImageJ software, as previously described (14). Image quantification for each sample consisted of the mean value from five independent and randomly-selected fields of view for each muscle section.

### Assessment of Muscle Function

Functional assessments of the EDL and diaphragm muscles were evaluated as previously described (17) by the University of Florida Physiological Assessment Core. Muscles of anesthetized mice were dissected and placed in physiological Ringer's solution gas equilibrated with 95% O₂/5% CO₂. After determining optimum length, muscles were subjected to three isometric contractions (stimulated at 120 Hz for 500 ms) to determine maximum tetanic tension (Po). Following these procedures, muscles were weighed, frozen embedded in OCT or snap-frozen, and stored at −80° C. until further use.

### Collagen Assay

Snap-frozen gastrocnemius samples were pulverized and transferred to pre-weighed 2 mL micro-centrifuge tubes. Tendon pieces were removed from samples during the pulverization procedure. Deionized H₂O was added to the sample at a volume of 10-times the sample mass, and the tissues were vigorously disrupted using a hand homogenizer (Benchmark Scientific Model D1000). A volume of 200 μL for each resulting sample homogenate was transferred to pre-weighed screw-top micro-centrifuge tubes, and samples were completely desiccated by overnight incubation at 65° C. to determine the dry mass of the sample to be assayed. This was important because of potential differences in tissue mass caused by edema that may affect assay results. Following tissue dry mass determination, the collagen content of the samples was measured using a colorimetric Total Collagen Assay kit (Biovision #K218), following the manufacturer's directions in a 96-well plate. Assay data were collected using a SpectraMax i3x multi-mode spectrophotometer (Molecular Devices) at a wavelength of 560 nm.

### Gene Expression

Gene expression analysis was conducted as previously described (17) using the following mouse-specific primers: Col1a2 (forward) 5′-ATG GTG GCA GCC AGT TTG AA-3′ (SEQ ID NO: 1) and (reverse) 5′-TCC AGG TAC GCA ATG CTG TT-3′ (SEQ ID NO: 2); Acta2 (forward) 5′-CTA CTG CCG AGC GTG AGA TTG TCC-3′ (SEQ ID NO: 3) and (reverse) 5′-GAG GGC CCA GCT TCG TCG TAT T-3′ (SEQ ID NO: 4); 116 (forward) 5′-AAC CAC GGC CTT CCC TAC TTC-3′ (SEQ ID NO: 5) and (reverse) 5′-TCT GGC TTT GTC TTT CTT GTT ATC-3′ (SEQ ID NO: 6); Tgfb1 (forward) 5′-GAC TCT CCA CCT GCA AGA CCA T-3′ (SEQ ID NO: 7) and (reverse) 5′-GGG ACT GGC GAG CCT TAG TT-3′ (SEQ ID NO: 8); Gapdh (forward) 5′-AGC AGG CAT CTG AGG GCC CA-3′ (SEQ ID NO: 9) and (reverse) 5′-TGT TGG GGG CCG AGT TGG GA-3′ (SEQ ID NO: 10). Relative gene expression quantification was performed using the ΔΔCt method with Gapdh as the normalization gene.

### Immunoblotting

Immunoblotting was performed as previously described (17) using the following primary antibodies: anti-dystrophin (1:1000; Abcam #15277), anti-NOX4 (1:2000; Abcam #13303), anti-fibronectin (1:2000; Sigma #F7387), and anti-GAPDH (1:2000; Santa Cruz #sc-25778). Quantified band signal intensities were measured using Image Studio Lite software (LI-COR Biosciences), normalized to GAPDH signal values, and reported relative to respective control samples.

### Cell Culture

Bone Marrow Derived Mesenchymal Stem Cells, human (BM-MSC, ATCC PCS-500-012) were cultured in medium composed of Mesenchymal Stem Cell Basal Medium foe Adipose, Umbilcal and Bone Marrow-derived MSCs (ATCC PCS-400030 and Mesenchymal Stem Cell Growth for Bone Marrow-derived MSCs (ATCC PCS-500-041). Cells were seeded at a density of 15,000 cells/well in a 96-well format.

To induce fibrogenic differentiation BM-MSCs were cultured in a medium composed by Dulbecco's Modified Eagle Medium, 2% horse serum (HS) (Sigma H1270) and 5-10 ng/ml TGF-beta-1 (PreproTech 100-21). Cells were cultured in basal media for 2 days before being switched to fibrogenic differentiation media for 5 days, after which they were taken down for downstream assays. Compound treatment was added when media was switched to fibrogenic differentiation media, cells were treated with 0.1% DMSO if not receiving compound.

### Immunocytochemistry

Cells were fixed in 4% PFA for 10 min at RT. Fixed cells were washed three times in 1×PBS and incubated in permeabilization solution (0.5% Triton X-100 in 1×PBS) for 10 min. Unspecific binding were saturated by incubating samples in blocking solution (5% donkey serum in 1×PBS) for 1 h. The cells were incubated at RT for 1 hour with gentle shaking in the presence of primary antibody against, alpha-smooth muscle actin (1:1000, Sigma™ A5228). The cells were washed three times with 0.05% Tween-20 in 1×PBS and incubated, for 1 hour at RT, with host-specific secondary antibodies in addition to counterstaining for Alexa Fluor Plus 750 Phalloidin (1:2000, Invitrogen™, A30105) and DAPI (1:2000). Primary and secondary antibodies were diluted according to the manufacturer's recommendations in blocking solution+0.1% Triton X-100. Finally, the cells were washed three times with 0.05% Tween-20 in 1×PBS, washed twice with 1×PBS and stored at 4° C. Images were acquired on a Thermo Scientific™ CellInsight™ CX7LED high content analysis (HCA) imaging instrument using a 10× objective. Nine fields per well were captured for quantification.

### Cellular Imaging Analysis

Columbus™ software (version 2.9.1, Perkin Elmer®) was used for analysis of acquired images. A custom analysis pipeline was constructed to quantify the percentage of myofibroblasts within the well. First, nuclei were segmented using “Method B” and intensity and morphology properties were calculated using standard settings. Second, viable nuclei were identified by gating on nuclei intensity, area, and roundness of the segmented objects. Next, a region of interest was then defined around the selected viable nuclei by expanding the outer boarder by −110%. STAR morphology and SER texture properties were calculated within the region of interest for the alpha-smooth muscle actin and phalloidin channels using the following parameters: profile width=4 px, scale=1 px, Kernel normalization. The calculated morphology and texture properties were used to train a linear classifier consisting of two classes, myofibroblasts and mesenchymal stem cells, by selecting >100 objects in each negative control (DMSO) and positive control (fibrogenic differentiation media) conditions. The linear classifier was then applied to the untrained test wells using a batch analysis to calculate the percentage of myofibroblasts within each image using a formula output (myofibroblasts:number of objects/viable nuclei:number of objects)*100. Results were averaged across the nine fields imaged per well.

### Statistical Analysis

Statistical analysis was performed using unpaired, two-tailed Welch's T-test [α=0.05; effect size reported as Cohen's d (d)] or ANOVA (Tukey post-hoc tests; α=0.05; effect size reports as η2). A P value less than 0.05 was considered significant. Values are displayed as box-and-whisker plots (depicting minimum and maximum values) or as bar graphs showing mean SEM.

### Example 2: NOX4 is an Anti-Fibrotic Target in D2.mdx Skeletal Muscle

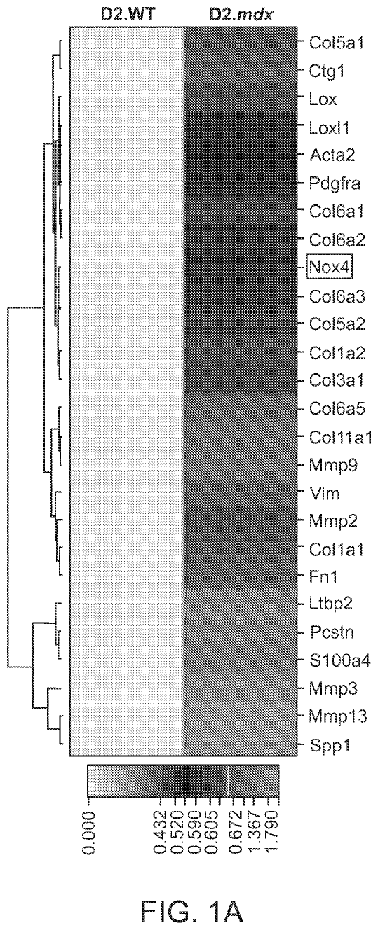

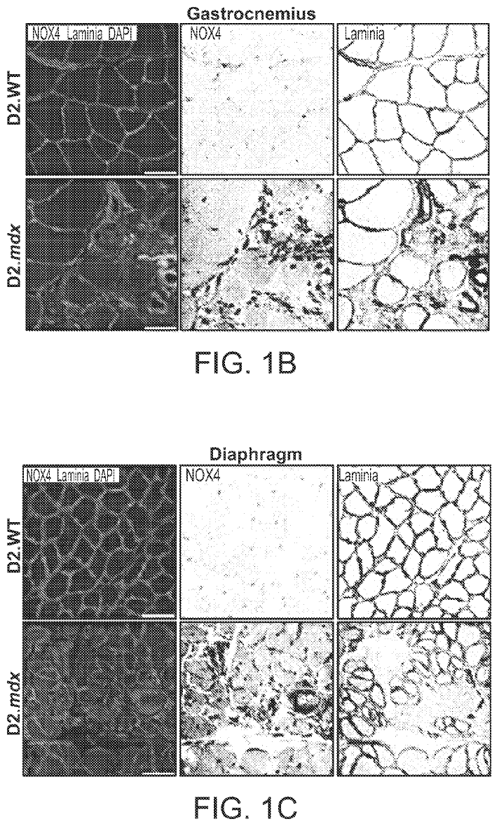

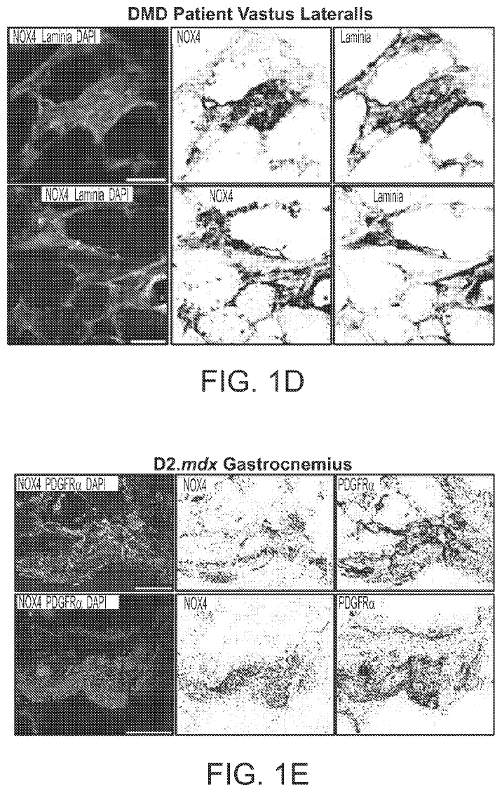

The heightened skeletal muscle fibrosis of D2.mdx mice made this emerging mouse model of Duchenne muscular dystrophy (DMD) better suited to investigate mechanisms contributing to muscle fibrosis than mdx mice on C57-based genetic backgrounds (14). To identify potential gene targets that may be exploited as anti-fibrotic therapies, a previously published transcriptomic dataset was queried for ECM/fibrosis-associated genes that were significantly upregulated in D2.mdx quadriceps muscle, relative to wild-type DBA/2J (D2.WT) values (p<0.05). Of 26 genes identified (FIG. 1A), the majority consisted of ECM components, including 10 isoforms of collagen, 4 members of matrix metallopeptidases (MMPs; a class of matrix remodeling enzymes), the collagen crosslinking enzymes Lox and Loxl1, and other ECM-related genes, including Fn1 (fibronectin), Lthp2, Postn (periostin), and Spp1 (osteopontin). Identified fibroblast-lineage markers included Pdgfra (platelet-derived growth factor receptor a (PDGFRα)), Ctgf, Vim (vimentin), S100a4, and Acta2 (α-smooth muscle actin (SMA)).

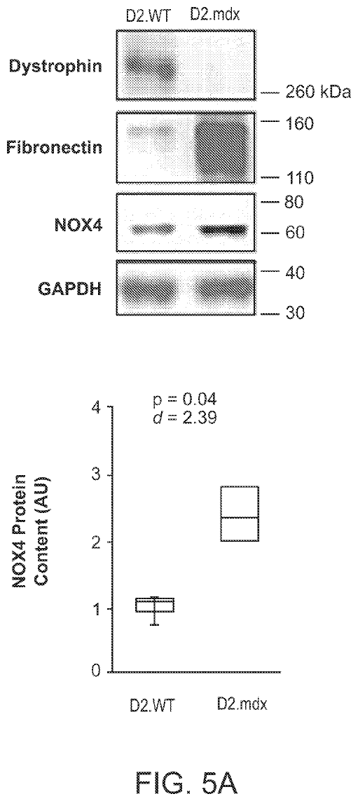

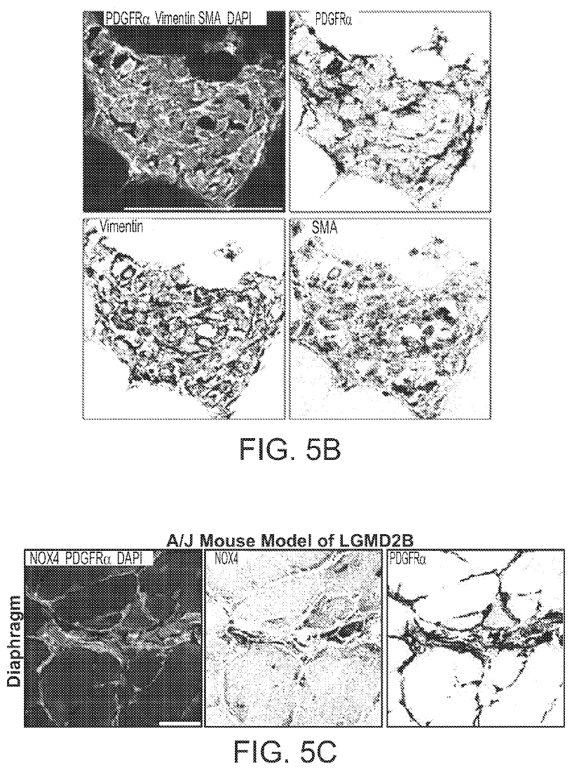

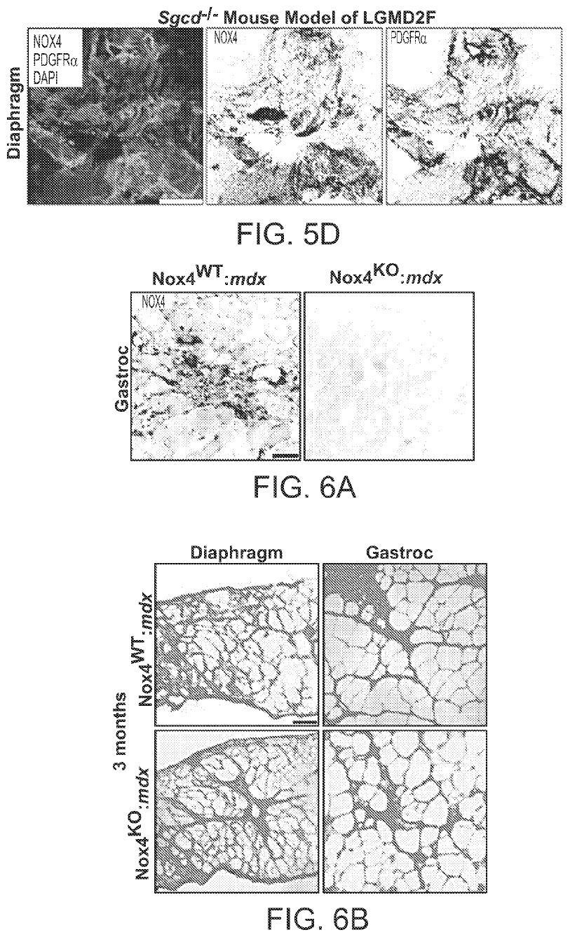

An interesting discovery amongst this query was identification in the DMD model of the increased expression of Nox4, which encodes the intracellular reactive oxygen species (ROS)-generating enzyme, NOX4. NOX4 has been identified as an anti-fibrosis target in several tissue types (21-28) but has not previously been associated with skeletal muscle fibrosis. Importantly, safe clinical-stage small-molecule inhibitors of this enzyme have been developed (29). Immunoblotting confirmed that NOX4 protein was elevated in D2.mdx skeletal muscle (FIG. 5A), which was localized at high concentrations in the interstitial regions between muscle fibers of D2.mdx mice, as shown in the gastrocnemius (FIG. 1B) and diaphragm (FIG. 1C). This NOX4 staining pattern was also found in the muscles of DMD patients (FIG. 1D). These NOX4-expressing cells observed in D2.mdx muscle also stained positive for PDGFRα (FIG. 1E), a marker of fibroblast-lineage cells, including fibro-adipogenic progenitor cells (FAP cells), fibroblasts, and myofibroblasts (30-33). Many of the PGDFRα⁺ cells found in NOX4-enriched interstitial regions also expressed vimentin and SMA (FIG. 5B), markers of myofibroblasts (34).

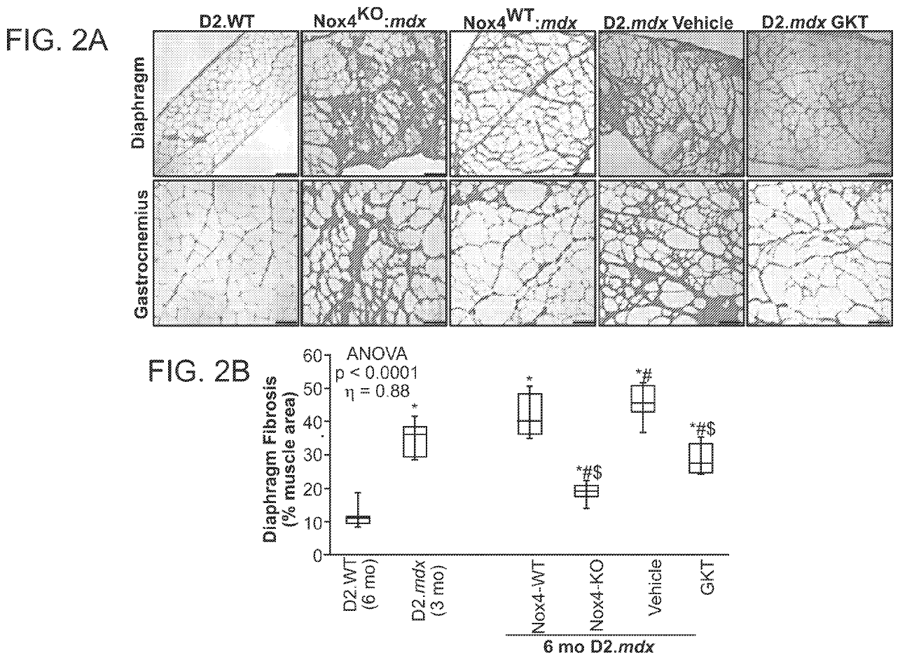

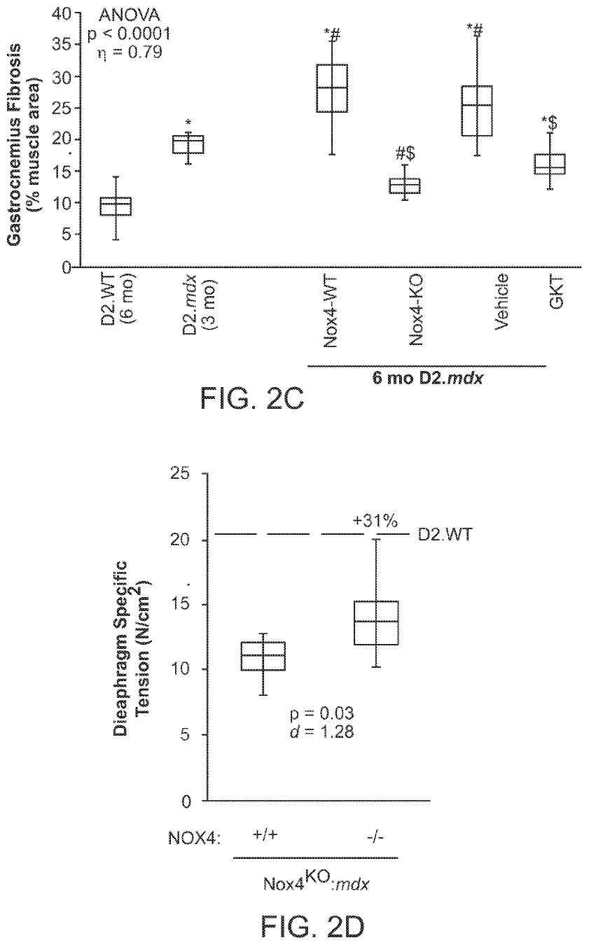

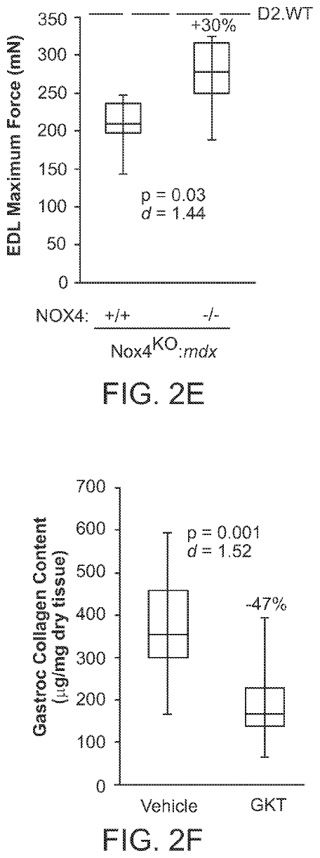

NOX4-expressing PDGFRα⁺ cells were also found in the muscles of mice that model limb-girdle MD (LGMD) 2B (dysferlinopathy; FIG. 5C) and 2F (6-sarcoglycan deficiency; (FIG. 5D)). Accordingly, these observations were not restricted to dystrophin-deficient muscle. This evidence suggested that NOX4 was involved in the development of muscle fibrosis in the MDs.

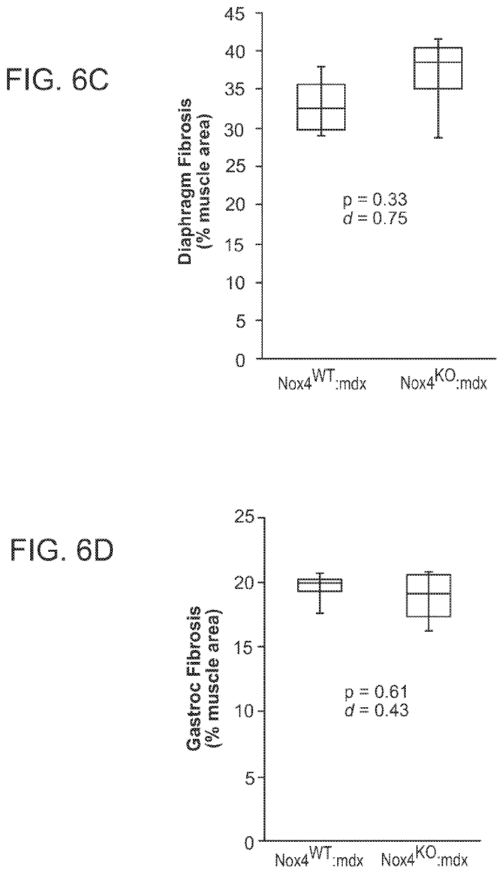

The role of NOX4 in the development of muscle fibrosis was directly tested by the generation of a Nox4 knockout mouse line on the D2.mdx background (Nox4^{KO} mdx). Wild-type littermates of this line (Nox4^{WT}:mdx) were indistinguishable from mice of the D2.mdx colony and exhibited NOX4 staining in the muscle interstitium, whereas Nox4^{KO}:mdx mice showed no NOX4 immunoreactivity (FIG. 6A). At 3 months of age, when D2.mdx mice began to show fibrosis during recovery from widespread muscle degeneration (14), Nox4^{WT}:mdx and Nox4^{KO}:mdx littermates showed no difference in muscle fibrosis, as assessed by histological evaluation using picrosirius red staining of the diaphragm and gastrocnemius (FIGS. 6B-6D). Therefore, NOX4 did not appear to affect ECM production associated with tissue repair from injury. At 6 months of age, when D2.mdx muscle displayed robust fibrotic progression independently of reparative activities (14), Nox4^{KO}:mdx diaphragms and gastrocnemius muscles exhibited significantly less fibrosis than those of the Nox4^{WT}:mdx littermates (FIGS. 2A-2C). While Nox4^{WT}:mdx muscles showed the expected increase in fibrosis from 3 months to 6 months, Nox4^{KO}:mdx muscles actually reduced muscle fibrosis during this timeframe. Thus, loss of NOX4 prevented the progressive accumulation of fibrosis in D2.mdx skeletal muscle, possibly by facilitating resolution of the post-injury response. NOX4-ablated diaphragms and extensor digitorum longus (EDL) muscles also exhibited significantly increased muscle function over those of Nox4^{WT}:mdx mice (FIGS. 2D and 2E), demonstrating that functional muscle was better maintained as fibrosis was reduced. Furthermore, pharmacological targeting of NOX4 phenocopied this anti-fibrotic effect of NOX4 ablation, as 3 months of treatment with the NOX1/4 inhibitor GKT831 (21, 29, 35) also reduced D2.mdx muscle fibrosis, compared to vehicle treatments, when evaluated at 6 months of age (FIGS. 2A-2C and 2F).

Collectively, these data indicated that targeting NOX4 for inhibition was an efficacious means to control the fibrotic replacement of dystrophic muscle.

### Example 3: NOX4 Inhibition Promoted Dystrophic Muscle Remodeling Through Clearance of Myofibroblasts

Myofibroblasts actively secrete ECM in tissues during repair and disease (20), and NOX4 was shown to be expressed by myofibroblasts in fibrotic lungs previously (24) and in dystrophic muscle herein (FIGS. 1C, 5D and 5E). Therefore, it was identified as likely that NOX4 inhibition exerted its anti-fibrotic effects primarily by targeting myofibroblasts. Consistent with such anti-fibrotic effects of NOX4 inhibition, myofibroblast numbers were identified as significantly reduced by both NOX4 ablation and GKT831 treatment (FIG. 3A), as assessed by labeling cells for PDGFRα, vimentin, and SMA. Additionally, these NOX4-targeting strategies were observed to have reduced the amount of periostin, an ECM component that was specifically expressed and secreted by myofibroblasts in muscle tissues (36), in D2.mdx muscle (FIG. 3B). These data indicated that the anti-fibrotic efficacy of NOX4 inhibition in dystrophic muscle was due to the clearance of myofibroblasts, thereby removing the source of pathological ECM deposition from the muscle.

Past studies have indicated that myofibroblasts may possess inhibited muscle regeneration capabilities (37-39). In agreement with suppressed regenerative capacity in disease-burdened D2.mdx muscle, Nox4^{WT}:mdx and vehicle-treated gastrocnemius muscles exhibited significantly reduced numbers of Pax7v satellite cells localized to their physiological niche between the sarcolemma and basal lamina of muscle fibers (FIG. 3C). However, a substantial number of Pax7v cells were found to be displaced into the dystrophic muscle interstitium (FIG. 3C), similar to what has been previously shown in DMD patient muscle (40) and murine cancer cachexia (41). In agreement with the beneficial remodeling of the dystrophic musculature by NOX4 targeting, NOX4 ablation and NOX1/4 inhibitor GKT831 treatments largely re-localized Pax7⁻ cells back to the periphery of muscle fibers (FIG. 3C). Furthermore, this return of Pax7⁺ cells to their physiological niche coincided with increases in muscle fibers having central nuclei (FIG. 3D) and expression of myosin X (FIG. 3E), both indicators of recent muscle regeneration (10, 42). Therefore, the beneficial remodeling of dystrophic muscle by NOX4 inhibition also appeared to include restoration of muscle regenerative capacity.

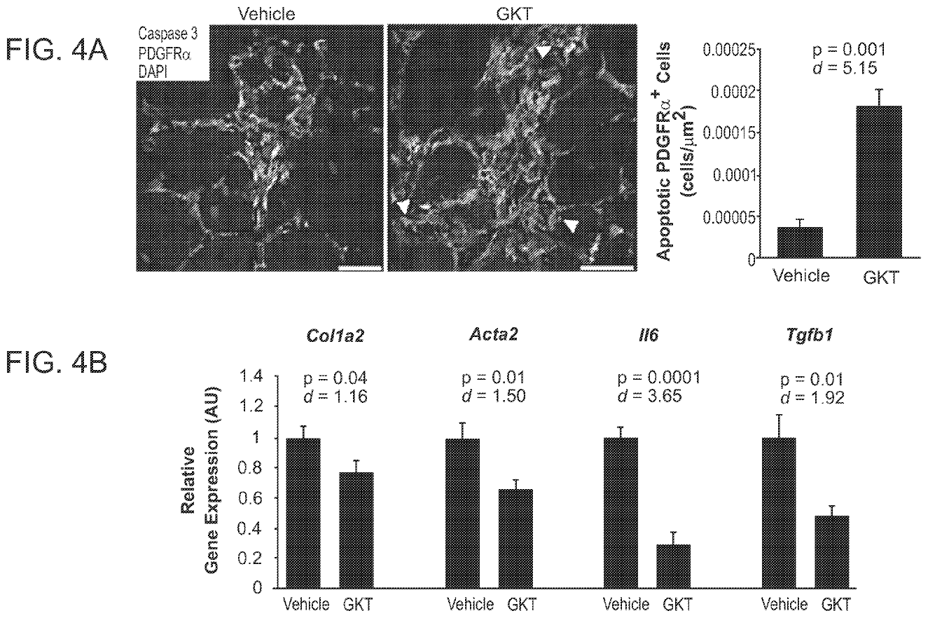

These data depicted a model whereby NOX4 targeting (via knockout or small molecule inhibitor) promoted the remodeling of dystrophic muscle via the clearance of myofibroblasts and alleviation of regeneration-inhibiting actions on satellite cells. Because the presumed mechanism attributed to myofibroblast clearance has been that of promoting their apoptosis (20), a short-term study was performed to investigate acute changes in muscle that result from NOX4 inhibition. Six-month-old D2.mdx mice, whose muscles exhibited substantial disease burden (14), received vehicle or GKT831 treatments for seven days. Following this dosing period, the gastrocnemius muscles of GKT831-treated mice exhibited significantly more PDGFRα⁺ cells showing positive staining for active Caspase-3 (FIG. 4A), an indicator of apoptosis. This demonstrated that the myofibroblast clearance achieved by NOX4 inhibition occurred, at least in part, via apoptosis. In agreement, muscles of GKT831-treated mice also exhibited decreased expression of the fibrosis/myofibroblast markers Col1a2 and Acta2, as well as the pro-fibrotic and anti-regeneration markers of Il6 and Tgfb1, compared to those of vehicle-treated mice (FIG. 4B). These data reinforced the notion that NOX4 inhibition promotes the remodeling of dystrophic muscle by inducing myofibroblast apoptosis.

### Example 4: Nox4 Inhibitors Effectively Induced FAP Cell-Mediated Skeletal Muscle Regeneration

Six small molecule inhibitors of NOX4—FTX1620, FTX7974, FTX 7981, FTX7983, FTX7995, and FTX8081—were initially tested in a mouse cell line modeling fibrogenic differentiation (cultured cells included FAP cells and bone marrow-derived mesenchymal stem cells (MSCs)) for impact upon myofibroblast content (reducing such fibrosis-depositing cells) in such cell line populations. The initial six NOX4 inhibitors were assessed in replicate experiments for both potency of myofibroblast reducing activity and an associated IC50 for the myofibroblast reduction phenotype was determined (Table 1).

TABLE 1
Proposed Compounds for Mouse PK/PD

| | | | | IC50 |
|---|---|---|---|---|
| | Generic Name/ | Reported | IC50 | Phenotype |
| FTX # | Asset Name | Potency | Phenotype | Replicate |
| FTX1620 | Setanaxib/ | Ki = | IC50 = | IC50 = |
| | GKT137831 | 110 nM | 77.3 nM | 126.3 nM |
| FTX7974 | UANOX048 | IC50 = | IC50 = | IC50 = |
| | | 2 uM | 357.2 nM | 332.7 nM |
| FTX7981 | GLX7013114 | IC50 = | IC50 = | IC50 = |
| | | 300 nM | 253.3 nM | 342.2 nM |
| FTX7983 | GLX481372 | IC50 = | IC50 = | IC50 = |
| | | 0.67 uM | 330.8 nm | 401.2 nm |
| FTX7995 | WO2017/ | IC50 = | IC50 = | IC50 = |
| | 192304 | 2 nM | 8.8 nM | 10.89 nM |
| FTX8018 | GKT136901 | Ki = | IC50 = | |
| | | 165 nM | 149.2 nM | |

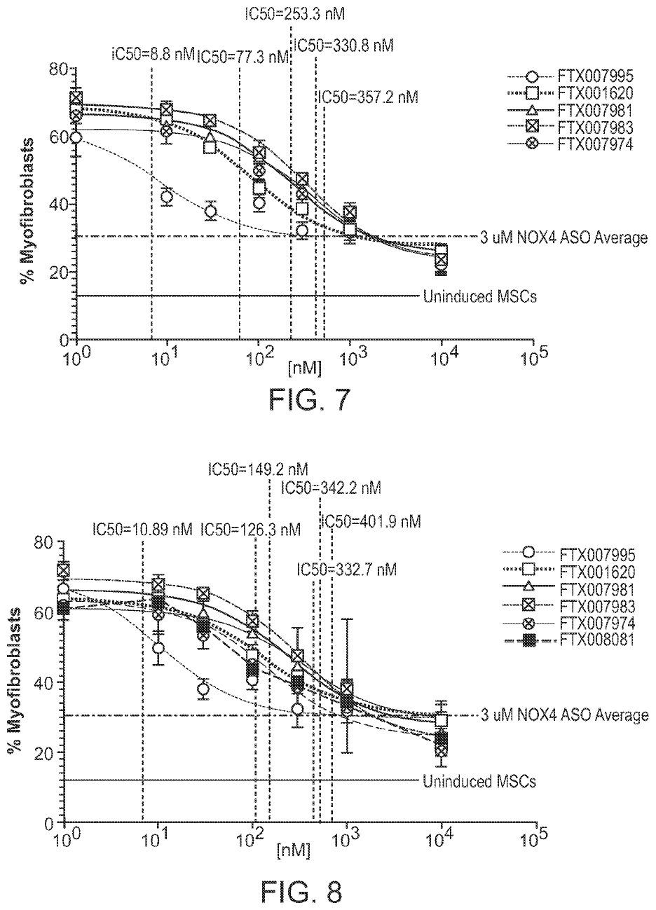

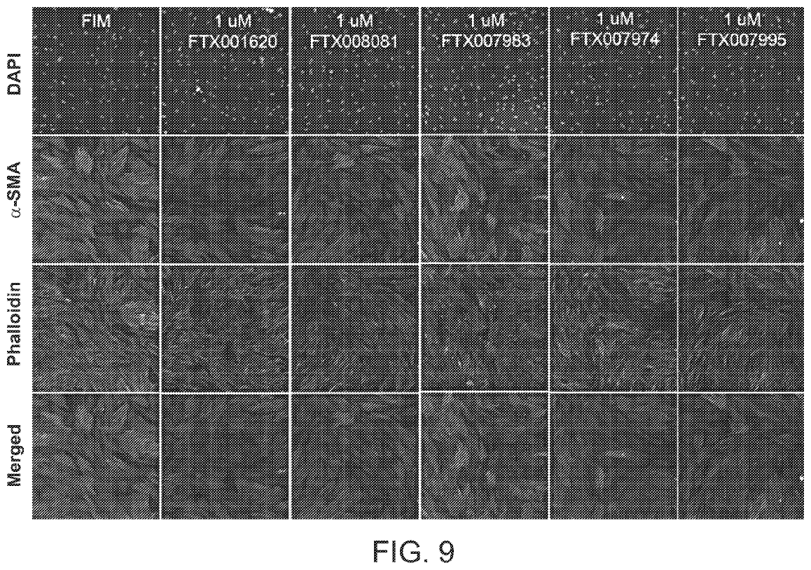

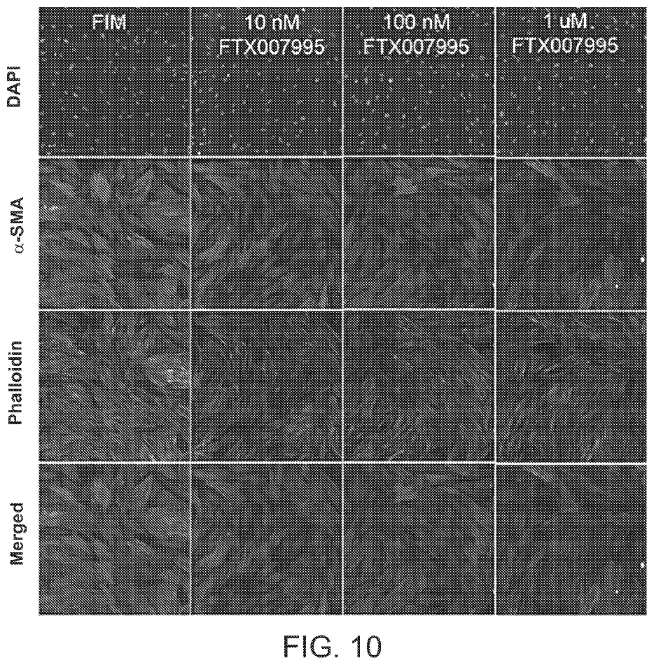

Each tested NOX4 inhibitor was identified to exhibit a dose-dependent reduction of myofibroblast content in treated populations of cells in culture (FIGS. 7 and 8), with observed IC50 phenotype values in the approximate 10 nM to 400 nM range. A panel of NOX4 inhibitors—FTX001620, FTX008081, FTX007963, FTX007974 and FTX007995—was examined for cellular impact in a cultured mesenchymal stem cell line. Treatment of assayed mesenchymal stem cells with each of the NOX4 inhibitors at a concentration of 1 μM resulted in significant phalloidin staining (a marker of F-actin activity), which indicated restoration of a pro-regenerative phenotype in treated cell populations (FIG. 9). The dose-dependent impact of one such NOX4 inhibitor, FTX007995 (aka FTX7995), was examined for cellular impact across a range of concentrations, by staining FTX007995-treated cells (at varying levels of FTX007995 treatment) for DAPI (nuclear marker) and phalloidin, while also detecting α-smooth muscle actin (α-SMA) by immunofluorescence. While cultured cells treated with a 10 nM concentration of the FTX007995 NOX4 inhibitor exhibited phalloidin staining, treatment of such cells with increased concentrations (100 nM and 1 μM) of the FTX007995 NOX4 inhibitor resulted in enhanced phalloidin staining (FIG. 10). NOX4 inhibitors were therefore surprisingly identified to restore treated cells to a pro-regenerative phenotype, such that muscle regeneration occurred in such NOX4i-treated mesenchymal cell populations.

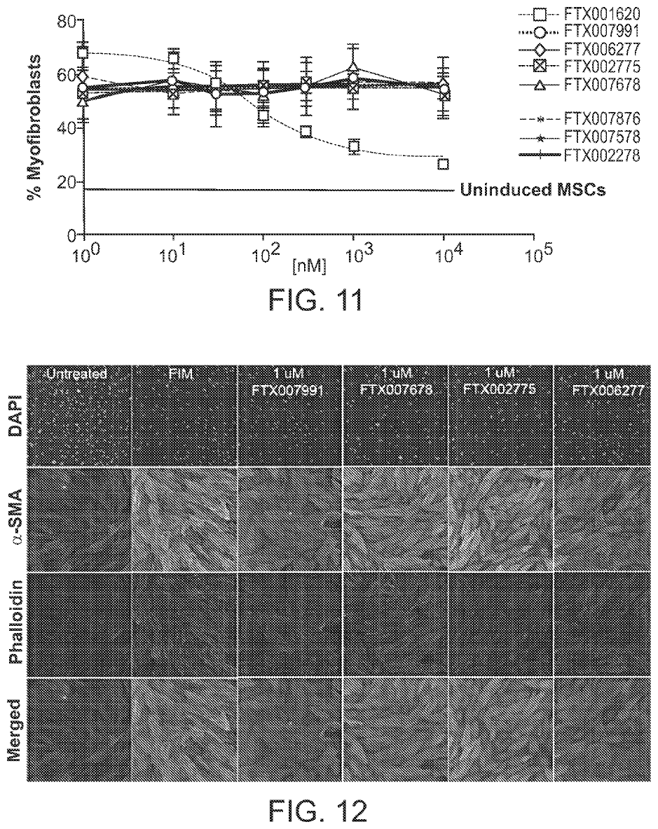

A variety of negative control compounds for the above-examined NOX4 inhibitors did not reduce myofibroblast differentiation in cell culture. Tested negative control compounds included FTX007991 (aka GLX481369, a redox active control lacking NOX4 inhibitory activity); FTX006277 (a compound possessing structural similarity to FTX1620); FTX002775 (a compound possessing structural similarity to FTX7971, though which can also be active); FTX007678 (a compound possessing structural similarity to FTX7995); FTX007876; FTX007578; and FTX002278. Concentration-response curves (CRCs) obtained for negative control compounds showed no reduction of myofibroblast content in treated cell populations at elevated concentrations of negative control compounds, in contrast to the NOX4-targeting FTX001620 positive control compound (FIG. 11). Administration of negative control compounds FTX007991, FTX007678, FTX002775, and FTX006277 to mesenchymal stem cells in culture failed to induce phalloidin staining (FIG. 12), which was further demonstrative of the NOX4-targeting specificity of the positive phalloidin staining results observed for above-tested NOX4 inhibitors FTX001620, FTX008081, FTX007963, FTX007974 and FTX007995. The ability of NOX4 inhibitors to restore a pro-regenerative phenotype to treated mesenchymal stem cells, such that muscle regeneration is induced therefore did not appear to be attributable to a more general redox/ROS (reactive oxygen species) scavenging mode of action for such compounds.

In view of the above results indicating that NOX4 inhibitor compounds might be used not only to reduce fibrosis in treated populations of mesenchymal stem cells but also could promote skeletal muscle regeneration via FAP cell activation, a number of additional, art-recognized NOX4 inhibitor compounds were tested for both potency and effect. Specifically, additional NOX4 inhibitors FTX007990, FTX007991, FTX007992 and FTX007993 were each assessed in replicate experiments for both potency of myofibroblast reducing activity and identification of an associated IC50 for the myofibroblast reduction phenotype (Table 2).

TABLE 2
Molecular Profiling Activity Template: Additional Compounds

| | | | | IC50 |
|---|---|---|---|---|
| | Generic Name/ | Reported | IC50 | Phenotype |
| FTX # | Asset Name | Potency | Phenotype | Replicate |
| FTX1620 | Setanaxib/ | Ki = | IC50 = | IC50 = |
| | GKT137831 | 110 nM | 77.3 nM | 126.2 nM |
| FTX7971 | GLX351322 | IC50 = | IC50 = | IC50 = |
| | | 5 uM | 4.025 μM | 5.21 μM |
| FTX7972 | VAS2870 | IC50 = | IC50 = | IC50 = |
| | | 2 nM | 889.2 nM | 898.4 nM |
| FTX7973 | APX-115 | Ki = | IC50 = | IC50 = |
| | | 0.63 uM | 637.75 nm | 780.7 nm |
| FTX7990 | Compound 7c | Ki = | IC50 = | IC50 = |
| | | 70 nM | 157.6 nM | 148.9 nM |

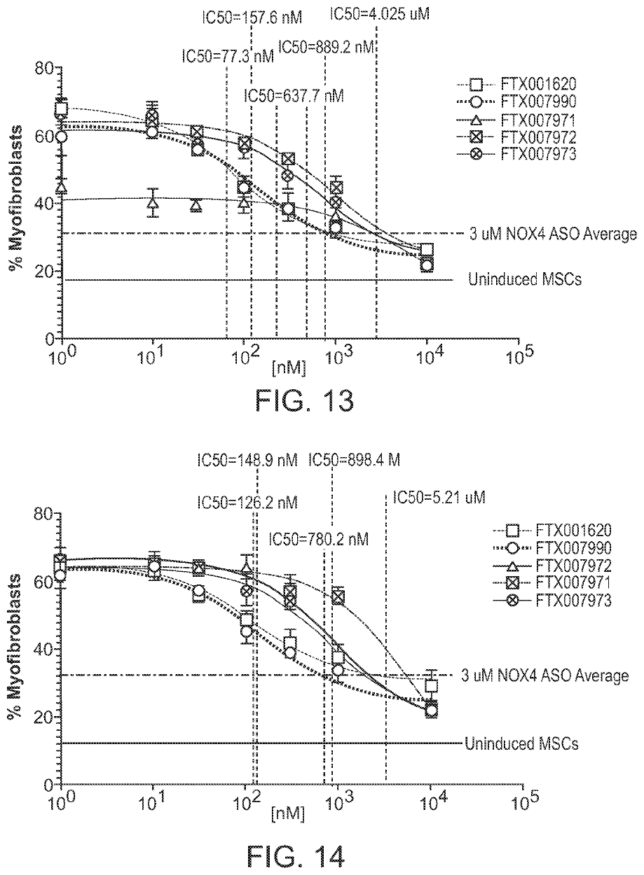

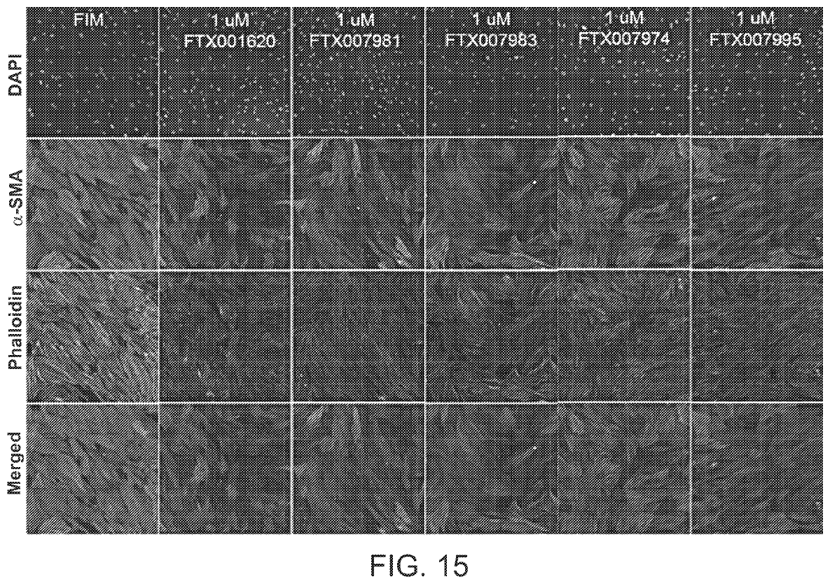

Additional NOX4 inhibitors FTX007990, FTX007991, FTX007992 and FTX007993 were each identified to exhibit a dose-dependent reduction of myofibroblast content in treated populations of cells in culture (FIGS. 13 and 14), with observed IC50 phenotype values in the approximate 150 nM to 5 μM range. When these same additional NOX4 inhibitors were administered to cells at 1 μM concentration and examined for cellular impact in a cultured mesenchymal cell line, significant phalloidin staining was observed (FIG. 15), which again indicated that NOX4 inhibition promoted restoration of a pro-regenerative phenotype to treated mesenchymal stem cells, apparently via activation of fibro-adipogenic progenitor cells (FAP cells).

### Example 5: Nox4 Inhibition Improved Myogenic Differentiation

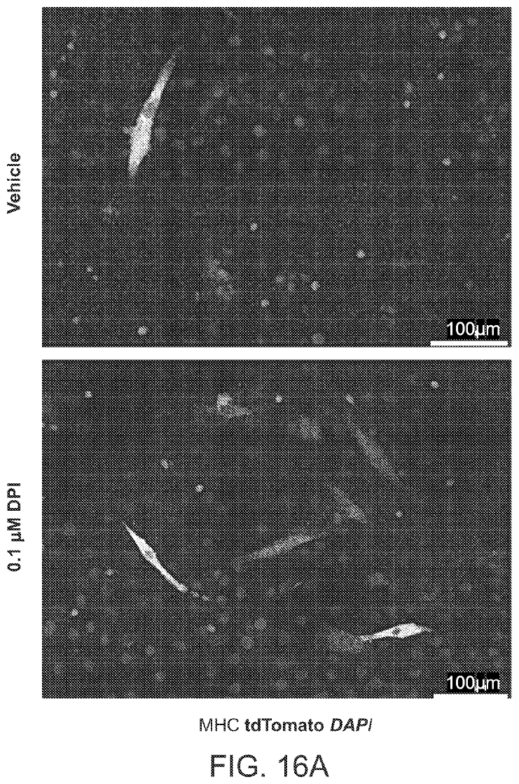

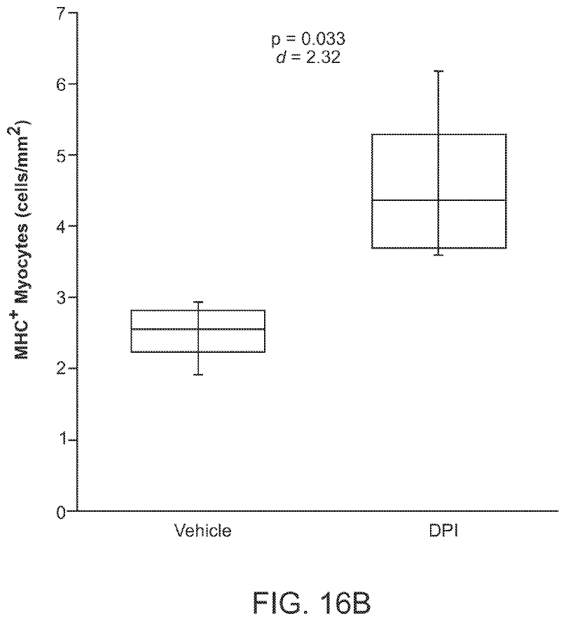

The impact of NOX4 inhibition on myogenic differentiation in myoblast-FAP co-cultures was investigated using primary myoblasts isolated from wild-type, uninjured mouse muscles and FAPs isolated from cardiotoxin-injured muscles of ROSA26-tdTomato mice (such mice contain cells that constitutively express the tdTomato fluorescent marker). Cells were mixed at a 10:1 myoblast:FAP ratio, plated on collagen-coated coverslips, and subjected to myogenic differentiation conditions in the presence of vehicle (DMSO; n=4) or 0.1 uM diphenyleneiodonium chloride (DPI; n=4). Following 3 days of differentiation, cells were fixed in 4% paraformaldehyde (PFA) and stained for myosin heavy chain (MHC; marker of myogenic differentiation). When imaged, myogenic differentiation of myoblasts was significantly increased in DPI-treated co-cultured myoblasts, as compared to vehicle-treated control co-cultures (FIG. 16A). MHC-positive myocytes were then quantified as number of cells per mm², and a statistically significant increase (p=0.033; d=2.32) in MHC-positive myocytes was demonstrated in DPI-treated co-cultures, relative to vehicle-treated control co-cultures (FIG. 16B). NOX4 inhibitors were therefore shown to have significantly promoted myogenic differentiation.

### REFERENCES

1. Mendell J R, et al. (2012) Evidence-based path to newborn screening for Duchenne muscular dystrophy. Ann Neurol 71(3):304-313.
2. Hoffman E P, Brown R H, Jr., & Kunkel L M (1987) Dystrophin: the protein product of the Duchenne muscular dystrophy locus. Cell 51(6):919-928.
3. Petrof B J, Shrager J B, Stedman H H, Kelly A M, & Sweeney H L (1993) Dystrophin protects the sarcolemma from stresses developed during muscle contraction. Proc Natl Acad Sci USA 90(8):3710-3714.
4. Willcocks R J, et al. (2016) Multicenter prospective longitudinal study of magnetic resonance biomarkers in a large duchenne muscular dystrophy cohort. Ann Neurol 79(4):535-547.
5. Charge S B & Rudnicki M A (2004) Cellular and molecular regulation of muscle regeneration.

### Physiol Rev 84(1):209-238.

6. Mauro A (1961) Satellite cell of skeletal muscle fibers. J Biophys Biochem Cytol 9:493-495.
7. Arnold L, et al. (2007) Inflammatory monocytes recruited after skeletal muscle injury switch into antiinflammatory macrophages to support myogenesis. J Exp Med 204(5):1057-1069.
8. Fry C S, Kirby T J, Kosmac K, McCarthy J J, & Peterson C A (2017) Myogenic Progenitor Cells Control Extracellular Matrix Production by Fibroblasts during Skeletal Muscle Hypertrophy. Cell Stem Cell 20(1):56-69.
9. Wosczyna M N, et al. (2019) Mesenchymal Stromal Cells Are Required for Regeneration and Homeostatic Maintenance of Skeletal Muscle. Cell Rep 27(7):2029-2035 e2025.
10. Hardy D, et al. (2016) Comparative Study of Injury Models for Studying Muscle Regeneration in Mice. PLoS One 11(1):e0147198.
11. McCarthy J J, et al. (2011) Effective fiber hypertrophy in satellite cell-depleted skeletal muscle. Development 138(17):3657-3666.
12. Patsalos A, et al. (2017) In situ macrophage phenotypic transition is affected by altered cellular composition prior to acute sterile muscle injury. J Physiol 595(17):5815-5842.
13. Dadgar S, et al. (2014) Asynchronous remodeling is a driver of failed regeneration in Duchenne muscular dystrophy. J Cell Biol 207(1):139-158.
14. Hammers D W, et al. (2020) The D2.mdx mouse as a preclinical model of the skeletal muscle pathology associated with Duchenne muscular dystrophy. Sci Rep 10(1):14070.
15. Fukada S, et al. (2010) Genetic background affects properties of satellite cells and mdx phenotypes. Am J Pathol 176(5):2414-2424.
16. Coley W D, et al. (2016) Effect of genetic background on the dystrophic phenotype in mdx mice. Hum Mol Genet 25(1):130-145.
17. Hammers D W, et al. (2020) Glucocorticoids counteract hypertrophic effects of myostatin inhibition in dystrophic muscle. JCI Insight 5(1).
18. Bernard K & Thannickal V J (2020) NADPH Oxidase Inhibition in Fibrotic Pathologies. Antioxidants & Redox Signaling 33(6):455-479.
19. Spurney C F, et al. (2008) Dystrophin-deficient cardiomyopathy in mouse: expression of Nox4 and Lox are associated with fibrosis and altered functional parameters in the heart. Neuromuscul Disord 18(5):371-381.
20. Hinz B & Lagares D (2019) Evasion of apoptosis by myofibroblasts: a hallmark of fibrotic diseases. Nature Reviews Rheumatology 16(1):11-31.
21. Gorin Y, et al. (2015) Targeting NADPH oxidase with a novel dual Nox1/Nox4 inhibitor attenuates renal pathology in type 1 diabetes. Am J Physiol Renal Physiol 308(11):F1276-1287.
22. Jha J C, et al. (2014) Genetic targeting or pharmacologic inhibition of NADPH oxidase nox4 provides renoprotection in long-term diabetic nephropathy. J Am Soc Nephrol 25(6):1237-1254.
23. Carnesecchi S, et al. (2011) A key role for NOX4 in epithelial cell death during development of lung fibrosis. Antioxid Redox Signal 15(3):607-619.
24. Hecker L, et al. (2009) NADPH oxidase-4 mediates myofibroblast activation and fibrogenic responses to lung injury. Nat Med 15(9):1077-1081.
25. Jarman E R, et al. (2014) An inhibitor of NADPH oxidase-4 attenuates established pulmonary fibrosis in a rodent disease model. Am J Respir Cell Mol Biol 50(1):158-169.
26. Jiang J X, et al. (2012) Liver fibrosis and hepatocyte apoptosis are attenuated by GKT137831, a novel NOX4/NOX1 inhibitor in vivo. Free Radic Biol Med 53(2):289-296.
27. Lan T, Kisseleva T, & Brenner D A (2015) Deficiency of NOX1 or NOX4 Prevents Liver Inflammation and Fibrosis in Mice through Inhibition of Hepatic Stellate Cell Activation. PLoS One 10(7):e0129743.
28. Somanna N K, et al. (2016) The Nox1/4 Dual Inhibitor GKT137831 or Nox4 Knockdown Inhibits Angiotensin-II-Induced Adult Mouse Cardiac Fibroblast Proliferation and Migration. AT1 Physically Associates With Nox4. J Cell Physiol 231(5):1130-1141.
29. Reutens A T, et al. (2020) A physician-initiated double-blind, randomised, placebo-controlled, phase 2 study evaluating the efficacy and safety of inhibition of NADPH oxidase with the first-in-class Nox-1/4 inhibitor, GKT137831, in adults with type 1 diabetes and persistently elevated urinary albumin excretion: Protocol and statistical considerations. Contemp Clin Trials 90:105892.
30. Joe A W, et al. (2010) Muscle injury activates resident fibro/adipogenic progenitors that facilitate myogenesis. Nat Cell Biol 12(2):153-163.
31. Uezumi A, Fukada S, Yamamoto N, Takeda S, & Tsuchida K (2010) Mesenchymal progenitors distinct from satellite cells contribute to ectopic fat cell formation in skeletal muscle. Nat Cell Biol 12(2):143-152.
32. Contreras 0, et al. (2019) Cross-talk between TGF-beta and PDGFRalpha signaling pathways regulates the fate of stromal fibro-adipogenic progenitors. J Cell Sci 132(19).
33. Li R, et al. (2018) Pdgfra marks a cellular lineage with distinct contributions to myofibroblasts in lung maturation and injury response. Elife 7:e36865.
34. Hu B & Phan S H (2013) Myofibroblasts. Curr Opin Rheumatol 25(1):71-77.
35. Aoyama T, et al. (2012) Nicotinamide adenine dinucleotide phosphate oxidase in experimental liver fibrosis: GKT137831 as a novel potential therapeutic agent. Hepatology 56(6):2316-2327.
36. Kanisicak O, et al. (2016) Genetic lineage tracing defines myofibroblast origin and function in the injured heart. Nat Commun 7:12260.
37. Hara M, et al. (2018) Periostin Promotes Fibroblast Migration and Inhibits Muscle Repair After Skeletal Muscle Injury. J Bone Joint Surg Am 100(16):e108.
38. Lorts A, Schwanekamp J A, Baudino T A, McNally E M, & Molkentin J D (2012) Deletion of periostin reduces muscular dystrophy and fibrosis in mice by modulating the transforming growth factor-beta pathway. Proc Natl Acad Sci USA 109(27):10978-10983.
39. Thooyamani A S & Mukhopadhyay A (2021) PDGFRalpha mediated survival of myofibroblasts inhibit satellite cell proliferation during aberrant regeneration of lacerated skeletal muscle. Sci Rep I1(1):63.
40. Kottlors M & Kirschner J (2010) Elevated satellite cell number in Duchenne muscular dystrophy. Cell Tissue Res 340(3):541-548.
41. He W A, et al. (2013) NF-kappaB-mediated Pax7 dysregulation in the muscle microenvironment promotes cancer cachexia. J Clin Invest 123(11):4821-4835.
42. Hammers D W, et al. (2021) Filopodia powered by class x myosin promote fusion of mammalian myoblasts. Elife 10:e72419.
43. Hecker L, et al. (2014) Reversal of persistent fibrosis in aging by targeting Nox4-Nrf2 redox imbalance. Sci Transl Med 6(231):231ra247.
44. Allen R E & Boxhorn L K (1987) Inhibition of skeletal muscle satellite cell differentiation by transforming growth factor-beta. J Cell Physiol 133(3):567-572.
45. Mazala D A, et al. (2020) TGF-beta-driven muscle degeneration and failed regeneration underlie disease onset in a DMD mouse model. JCI Insight 5(6):e135703.
46. Duan D (2018) Systemic AAV Micro-dystrophin Gene Therapy for Duchenne Muscular Dystrophy. Mol Ther 26(10):2337-2356.
47. England S B, et al. (1990) Very mild muscular dystrophy associated with the deletion of 46% of dystrophin. Nature 343(6254):180-182.
48. Barp A, et al. (2017) Muscle MRI and functional outcome measures in Becker muscular dystrophy. Sci Rep 7(1):16060.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize or be able to ascertain using no more than routine experimentation, many equivalents to the specific embodiments of the disclosure described herein. Such equivalents are intended to be encompassed by the following claims.

1. A method for activating fibro-adipogenic progenitor (FAP) cells to increase muscle regeneration in a subject, the method comprising administering a NADPH oxidase-4 (NOX4) inhibitor to the subject in an amount sufficient to activate FAP cells of the subject to increase muscle regeneration, thereby activating FAP cells to increase muscle regeneration in the subject.

2. The method of claim 1, further comprising monitoring FAP cell-mediated muscle regeneration in the subject, optionally wherein said monitoring comprises measurement of one or more biomarkers for muscle regeneration in the subject, assessment of fibrofatty infiltrate in the subject and/or assessment of lean muscle volume in the subject, optionally wherein the assessment of lean muscle volume in the subject is via magnetic resonance imaging (MRI), the assessment of fibrofatty infiltrate in the subject comprises measurement of muscle fat infiltration or fibrosis in the subject by MRI or multispectral optoacoustic tomography, and/or wherein the one or more biomarkers for muscle regeneration are selected from the group consisting of myogenic transcription factors (e.g., MyoD, Myf5, MyoG), satellite cell factors (e.g., Pax7), and developmental myosin heavy chains (e.g., MYH3/MYH8).

3. The method of claim 1, wherein the NOX4 inhibitor is selected from the group consisting of a small molecule, a peptide/nucleic acid aptamer, an antibody (or antibody fragment) of a NOX4 interaction partner which behaves as an antagonist of NOX4 function, and an inhibitory and/or antisense RNA (e.g. siRNA, shRNA, ASO, etc.).

4. The method of claim 3, wherein the small molecule inhibitor of NOX4 is selected from the group consisting of steanaxib (GKT137831), GKT136901, GLX351322, APX-115, compound 7c, VAS2870, GLX481372, GLX7013114, UANox048, Ex. 101, thioridazine, prochlorproazine, chlorpromazine, fluphenazine, perhenazine, promazine, VAS3947, perhexiline, suramin, ebselin, celastrol, ML090, imipramin blue, imipramin hydrochloride, 3-methyl-1-phenyl-pyrazoline-5-one, GSK2795039, DPI, and a sulfonylurea compound.

5. The method of claim 4, wherein the small molecule inhibitor of NOX4 is administered in combination with a nucleic acid therapeutic, optionally wherein the nucleic acid therapeutic is a NOX4-targeting nucleic acid therapeutic.

6. The method of claim 1, wherein the subject has or is at risk of developing muscular dystrophy (MD), a myopathy, a myositis, and spinal muscular atrophy (SMA), optionally wherein the MD is selected from the group consisting of Facioscapulohumeral muscular dystrophy (FSHD), Duchenne muscular dystrophy (DMD), limb-girdle muscular dystrophies (LGMD), and congenital muscular dystrophy (CMD).

7. The method of claim 1, wherein the subject is human.

8. The method of claim 1, wherein the subject is a mouse, optionally a D2-mdx mouse.

9. The method of claim 1, wherein the muscle regeneration occurs by a process of satellite cell-mediated muscle repair.

10. The method of claim 1, wherein the muscle regeneration occurs at a level of at least two-fold higher in a subject administered the NOX4 inhibitor, as compared to an appropriate control.

11. A method selected from among:
A method for activating FAP cells in vitro to increase muscle regeneration, the method comprising contacting the FAP cells with a NOX4 inhibitor in an amount sufficient to activate FAP cells to increase muscle regeneration;
A method for treating a muscle in a subject, the method comprising administering a NOX4 inhibitor to the subject in an amount sufficient to promote muscle regeneration in the subject, thereby treating the muscle in the subject;
A method for promoting muscle regeneration in a muscle of a subject, the method comprising administering a NOX4 inhibitor to the subject in an amount sufficient to promote muscle regeneration in the subject; and
A method comprising use of a NOX4 inhibitor in the preparation of a medicament for inducing muscle regeneration in a subject, wherein the medicament is prepared to be administered in an amount sufficient to induce muscle regeneration in the subject by activating FAP cells of the subject.

12. The method of claim 11, wherein the FAP cells are in a skeletal muscle organoid, optionally wherein the skeletal muscle organoid is a human skeletal muscle organoid.

13. The method of claim 11, further comprising monitoring the FAP cells for activation of muscle regeneration, optionally comprising monitoring one or more biomarkers of muscle regeneration in FAP cell-associated myogenic cells, optionally wherein the one or more biomarkers of muscle regeneration in the FAP cell-associated myogenic cells are selected from the group consisting of myogenic transcription factors (e.g., MyoD, Myf5, MyoG), satellite cell factors (e.g., Pax7), and developmental myosin heavy chains (e.g., MYH3/MYH8).

14. The method of claim 11, wherein the NOX4 inhibitor is selected from the group consisting of a small molecule, a peptide/nucleic acid aptamer, an antibody (or antibody fragment) a fragment of a NOX4 interaction partner which behaves as an antagonist of NOX4 function, and an inhibitory and/or antisense RNA (e.g., siRNA, shRNA, ASO, etc.).

15. The method of claim 14, wherein the small molecule inhibitor of NOX4 is selected from the group consisting of steanaxib (GKT137831), GKT136901, GLX351322, APX-115, compound 7c, VAS2870, GLX481372, GLX7013114, UANox048, Ex. 101, thioridazine, prochlorproazine, chlorpromazine, fluphenazine, perhenazine, promazine, VAS3947, perhexiline, suramin, ebselin, celastrol, ML090, imipramin blue, imipramin hydrochloride, 3-methyl-1-phenyl-pyrazoline-5-one, GSK2795039, DPI, and a sulfonylurea compound.

16. The method of claim 15, wherein the small molecule inhibitor of NOX4 is administered in combination with a nucleic acid therapeutic, optionally wherein the nucleic acid therapeutic is a NOX4-targeting nucleic acid therapeutic.

17. A pharmaceutical composition for inducing muscle regeneration in a subject, the pharmaceutical composition comprising a NOX4 inhibitor in an amount sufficient to induce muscle regeneration in the subject.

18. The pharmaceutical composition of claim 17, wherein the NOX4 inhibitor is present at a concentration capable of preventing at least 50% of myofibroblast differentiation in a subject administered the pharmaceutical composition, as compared to an appropriate control, optionally wherein the appropriate control comprises in vitro assessment of myofibroblast differentiation.

19. The pharmaceutical composition of claim 17, wherein the NOX4 inhibitor is present at a concentration capable of inducing at least 50% greater myogenic differentiation in a subject administered the pharmaceutical composition, as compared to an appropriate control, optionally wherein the appropriate control comprises in vitro assessment of myogenic differentiation.

20-22. (canceled)


SEQUENCE LISTING (10 sequences)

SEQ ID NO: 1 - DNA, 20 bp, synthetic construct
  source 1..20: mol_type = other DNA
atggtggcagccagtttgaa

SEQ ID NO: 2 - DNA, 20 bp, synthetic construct
  source 1..20: mol_type = other DNA
tccaggtacgcaatgctgtt

SEQ ID NO: 3 - DNA, 24 bp, synthetic construct
  source 1..24: mol_type = other DNA
ctactgccgagcgtgagattgtcc

SEQ ID NO: 4 - DNA, 22 bp, synthetic construct
  source 1..22: mol_type = other DNA
gagggcccagcttcgtcgtatt

SEQ ID NO: 5 - DNA, 21 bp, synthetic construct
  source 1..21: mol_type = other DNA
aaccacggccttccctacttc

SEQ ID NO: 6 - DNA, 24 bp, synthetic construct
  source 1..24: mol_type = other DNA
tctggctttgtctttcttgttatc

SEQ ID NO: 7 - DNA, 22 bp, synthetic construct
  source 1..22: mol_type = other DNA
gactctccacctgcaagaccat

SEQ ID NO: 8 - DNA, 20 bp, synthetic construct
  source 1..20: mol_type = other DNA
gggactggcgagccttagtt

SEQ ID NO: 9 - DNA, 20 bp, synthetic construct
  source 1..20: mol_type = other DNA
agcaggcatctgagggccca

SEQ ID NO: 10 - DNA, 20 bp, synthetic construct
  source 1..20: mol_type = other DNA
tgttgggggccgagttggga